SEQUENCE LISTING (4 sequences)

SEQ ID NO: 1 - protein, 7 aa, Artificial Sequence
  Peptide substrate
KPQGLEA

SEQ ID NO: 2 - protein, 7 aa, Artificial Sequence
  Peptide substrate
PQGLEAK

SEQ ID NO: 3 - protein, 6 aa, Artificial Sequence
  Peptide substrate
PQGLAK

SEQ ID NO: 4 - protein, 7 aa, Artificial Sequence
  Peptide substrate
PQGLKAK

(19) United States
(12) Patent Application Publication
(10) Pub. No.: US 2008/0176263 A1
(43) Pub. Date: Jul. 24, 2008

(54) Materials and Methods for Efficient and Accurate Detection of Analytes

(75) Inventors: Gregory Schultz, Gainesville, FL (US); John I. Azeke, Gainesville, FL (US); Daniel J. Gibson, Gainesville, FL (US); Olajompo Busola Moloye, Gainesville, FL (US); Priscilla Lorraine Phillips, Gainesville, FL (US); Weihong Tan, Gainesville, FL (US); Christopher D. Batich, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 11/952,943

(22) Filed: Dec. 7, 2007

Related U.S. Application Data
(60) Provisional application No. 60/873,477, filed on Dec. 7, 2006.

(51) Int. Cl. C12Q 1/37; C12M 1/34; G01N 21/75

(52) U.S. Cl. 435/23; 435/287.1; 436/172; 435/287.9

(57) ABSTRACT

The present invention provides diagnostic methods and devices that can be used to assay a medium, such as tissue in vivo or a sample in vitro (e.g. biological sample or environmental sample), in order to determine the presence, quantity, and/or concentration ratio of one or more target analytes.

### BACKGROUND OF THE INVENTION

The rapid and accurate detection of target molecules and microorganisms is critical for many areas of research, environmental assessment, food safety, medical diagnosis, and warfare.

Important features for a diagnostic technique to be used for the detection of analytes are specificity, speed, and sensitivity. Time constraints and ease of on-site analysis can be major limitations. For example, in the case of diagnostics for microorganisms, many detection methods rely on the ability of microorganisms to grow into visible colonies over time in special growth media, which may take about 1-5 days. Moreover, detection of trace amounts of bacteria typically requires amplification or enrichment of the target bacteria in the sample. These methods tend to be laborious and time consuming.

In vitro diagnostic assays of biological compounds have become routine for a variety of applications, including medical diagnosis, forensic toxicology, pre-employment and insurance screening, and food borne pathogen testing. Most systems can be characterized as having three key components: a probe that recognizes the target analyte(s) with a high degree of specificity; a reporter that provides a signal that is qualitatively or quantitatively related to the presence of the target analyte; and a detection system capable of relaying information from the reporter to a mode of interpretation. The probe (e.g., antibody, nucleic acid sequence, or enzyme product/activity) should interact uniquely and with high affinity to the target analyte, but not with non-targets. In order to minimize false positive responses, it should not react with non-targets.

The label is often directly or indirectly coupled (conjugated) to the probe, providing a signal that is related to the concentration of analyte upon completion of the assay. The label should not be subject to signal interference from the surrounding matrix, either in the form of signal loss from extinction or by competition from non-specific signal (noise) from other materials in the system.

The detector is usually a device or instrument used to determine the presence of the reporter (and therefore analyte) in the sample. Ideally, the detector should provide an accurate and precise quantitative scale for the measurement of the analyte. In rapid on-site tests, such as pregnancy tests, the detection instrument is the human eye and the test results are qualitative (positive or negative).

Immunochromatographic assays for detecting various analytes of interest have been known for some time. Some of the more common assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbiol, 2001, 39:3495-3498). Human immunodeficiency virus (HIV) has been detected rapidly in pooled blood using a similar assay (Soroka, S. D. et al. J Clin Virol, 2003, 27:90-96). A nitrocellulose membrane card has also been used to diagnose schistosomiasis by detecting the movement and binding of nanoparticles of carbon (van Dam, G. J. et al. J Clin Microbiol, 2004, 42:5458-5461).

The need for more sensitive yet simple optical-based bioanalytical techniques can be addressed by coupling nanotechnology with traditional bioanalytical methods for the detection of bacteria, virus, antibodies, DNA hybridization, and other molecular species needing sensitive recognition. Fluorescent nanoparticles have been developed (Zhao, X. et al. Proc Natl Acad Sci USA, 2004, 101:15027-15032; Qhobosheane, M. et al. Analyst, 2001, 126:1274-1278; Santra, S. et al. Anal Chem, 2001, 73:4988-4993; Santra, S. et al. Advanced Materials, 2005, 17:2165-2169; Wang, L. et al. Nano Letters, 2005, 5:37-43; Zhao, X. J. et al. Advanced Materials, 2004, 16:173-+; Santra, S. et al. Journal of Biomedical Optics, 2001, 6:160-166; Santra, S. et al. Chemical Communications, 2004, 2810-2811; Bagwe, R. P. et al. Langmuir, 2004, 20:8336-8342). Such nanoparticles have been utilized for sensitive bioassays, including biomarking (Santra, S. et al. Anal Chem, 2001, 73:4988-4993; Lian, W. et al. Analytical Biochemistry, 2004, 334:135-144), biosensors (Santra, S. et al. Journal of Biomedical Optics, 2001, 6:160-166; Tapec, R. et al. Journal of Nanoscience and Nanotechnology, 2002, 2:405-409), and immunological (Lian, W. et al. Analytical Biochemistry, 2004, 334:135-144) based detection. When compared to fluorescent dye molecules, the dye-doped nanoparticles provide enhanced signal because the bio-recognition event is linked with 10,000 (Zhao, X. J. et al. Journal of the American Chemical Society, 2003, 125:11474-11475) times more dye molecules.

Some of the studies that have been conducted with these new materials include their preparation, characterization (Zhao, X. et al. Proc Natl Acad Sci USA, 2004, 101:15027-15032; Qhobosheane, M. et al. Analyst, 2001, 126:1274-1278; Santra, S. et al. Anal Chem, 2001, 73:4988-4993; Santra, S. et al. Advanced Materials, 2005, 17:2165-2169; Wang, L. et al. Nano Letters, 2005, 5:37-43; Zhao, X. J. et al. Advanced Materials, 2004, 16:173-176; Santra, S. et al. Journal of Biomedical Optics, 2001, 6:160-166; Santra, S. et al. Chemical Communications, 2004, 2810-2811; Bagwe, R. P. et al. Langmuir, 2004, 20:8336-8342) surface modification, and bioconjugation (Zhao, X. et al. Proc Natl Acad Sci USA, 2004, 101:15027-15032; Qhobosheane, M. et al. Analyst, 2001, 126:1274-1278; Wang, L. et al. Nano Letters, 2005, 5:37-43; Santra, S. et al. Chemical Communications, 2004, 2810-2811; Lian, W. et al. Analytical Biochemistry, 2004, 334:135-144; Zhao, X. J. et al. Journal of the American Chemical Society, 2003, 125: 11474-11475) of dye-doped silica nanoparticles for bioanalysis, specifically for DNA analysis (Zhao, X. J. et al. Journal of the American Chemical Society, 2003, 125:11474-11475) and pathogenic bacteria detection (Zhao, X. et al. Proc Natl Acad Sci USA, 2004, 101:15027-15032).

Proteases are implicated in disparate pathologies including: virulence factors that facilitate infectious diseases (Matayoshi, E. D. et al. Science, 247 (February 1990): 954-958; Sham, H. L. et al. Journal of Medicinal Chemistry, 39, no. 2 (1996): 392-397; Sham, H. L. et al. Antimicrobial Agents and Chemotherapy, 42, no. 12 (1998): 3218-3224), metastasis of cancerous cells (McCawley, L. J. and L. M. Matrisian Current Opinion in Cell Biology, 13 (2001): 534-540), tissue damage in periodontal disease (Sandholm, L. Journal of Clinical Periodontology, 13, no. 1 (1986): 19-26), complications in pregnancy (Locksmith, G. J. et al. Am J Obstet Gynecol, 184, no. 2 (January 2001): 159-164), tissue destruction in inflamed joints (Cunnane, G. et al. Arthritis & Rheumatism, 44, no. 8 (2001): 1744-1753), and destruction of pro-healing factors and nascent tissue in chronic, non-healing, wounds (Ladwig, G. P. et al. Wound Repair and Regeneration, 10 (2002): 26-37; Trengove, N. J. et al. Wound Repair and Regeneration, 7 (1999): 442-452; Yager, D. R. et al. Wound Repair and Regeneration, 5 (1997): 23-32).

Studies of proteases in diseases have employed tests from one of two (or a combination of the two) classes: molecular presence-based tests, or catalytic activity-based tests. A common molecular presence-based test would be an immuno-detection assay where the protease of interest is isolated from the rest of the sample and antibodies that specifically recognize that protease are labeled with a detectable agent. The other class, catalytic activity-based, does not just measure whether the molecule (or the portion of the molecule that an antibody recognizes) is present, it measures how active the molecule is in the given conditions. A clinical example of the catalytic activity based class is a glucose oxidase test used by diabetics.

Currently, three protease activity based assays are in common laboratory use: the zymogram (Quesada, A. R. et al. Clin. Exp. Metastasis, 15 (1997): 26-32), the thiopeptolide continuous calorimetric assay (Stein, R. L. and M. Izquierdo-Martin Archives of Biochemistry and Biophysics, 308, no. 1 (January 1994): 274-277; Oxford Biomedical Research. Colorimetric Drug Discovery Assay for Matrix Metalloproteinase-7, Product Brochure, Oxford, Mich.: Oxford Biomedical Research, 2005 Oxford Biomedical Research. Colorimetric Drug Discovery Assay for Matrix Metalloproteinase-7, Product Brochure, Oxford, Mich.: Oxford Biomedical Research, 2005; Rosa-Bauza, Y. T. et al. ChemBioChem, 8 (2007): 981-984), and the fluorescence resonance energy transfer (FRET) continuous fluorometric assay (Fairclough, R. H. and C. R. Cantor Methods in Enzymology, 48 (1978): 347-379; Stryer, L. Annu Rev Biochem, 47 (1978): 819-846; Yaron, A. et al. Analytical Biochemistry, 95, no. 1 (May 1979): 228-235; Matayoshi, E. D. et al. Science, 247 (February 1990): 954-958; Beekman, B. et al. FEBS Letters, 390, no. 2 (1996): 221-225; Knäuper, V. et al. The Journal of Biological Chemistry, 271, no. 3 (January 1996): 1544-1550).

The zymogram is usually used when analyzing mixtures of proteases since it first resolves the different proteases by mass and then measures their activity. The thiopeptolide assay is used by suppliers of proteases to verify/guarantee a basic level of protease activity in the supplied sample (Calbiochem Data Sheet PF024 Rev. 25-September-06 RFH) (Biomol Product Data Catalog No.: SE-244).

Many currently marketed rapid, point-of-care diagnostic technologies are limited by their analytical sensitivity or by the number of analytes detected in a single assay.

### BRIEF SUMMARY OF THE INVENTION

The present invention provides diagnostic methods and devices that can be used to assay a medium, such as tissue in vivo or a sample in vitro (e.g., biological sample or environmental sample), in order to determine the presence, quantity, and/or concentration ratio of one or more target analytes.

The analytes detected according to the subject invention can be biochemical markers of health that can be used to direct therapy or prophylaxis. Thus, the device and method of the invention can be of great benefit when diagnosing a pathological condition that has one or more biochemical markers. For example, a non-healing (chronic) wound is marked by the imbalance of several biological regulators, such as cytokines, proteases, and protease inhibitors, representing potential target analytes for the assays of the present invention. In one embodiment, the present invention is particularly useful for differential assays, in which a comparison between the amounts of multiple target molecules in the same sample or site is of interest.

Advantageously, in certain embodiments, the subject invention provides assays that can be self-contained in a single unit. This facilitates conducting assays in the field and, in the case of healthcare, at the point of care.

In an embodiment that is specifically exemplified herein, the subject invention provides assays that can be used to determine and/or monitor the status of a wound. The assays are quick and easy-to-use. In specific embodiments the assay can be carried out by, for example, a nurse utilizing either no instrumentation or only minimal instrumentation. In one embodiment, information about the status of a wound can be readily, easily and reliably generated in 10 minutes or less. Information about the wound can include, but is not limited to, protease activity, bacterial presence, and/or nitric oxide status.

In a preferred embodiment of the subject invention the assay is a soluble-substrate based assay. Particularly preferred assays as described herein include FRET and calorimetric assays. Other assay formats, including those with a solid substrate, may also be utilized as described herein.

The subject invention also provides sample collection methodologies which, when combined with the assays of the subject invention, provide a highly advantageous system for analyte evaluation in a wide variety of settings. In one embodiment, a “swab-in-a-straw” collection and assay system can be utilized as described herein.

A further assay format utilizes a thin film for the detection of collagenase and/or other enzymes. In this context, the thin film can be, or can comprise, gelatin for the purpose of detecting collagenase. Alternative enzyme assays can utilize albumin or casein as the thin film.

Target analytes can be endogenous or exogenous to the medium to be assayed. For example, a target molecule can be a protease inhibitor that is normally found in the tissue or an anatomical sample site. In another embodiment, a target molecule is exogenous to the tissue or sample site, e.g., having been administered to the subject for the purpose of treatment or prophylaxis. For example, proteases regulate many physiological processes by controlling the activation, synthesis and turnover of proteins. Many small molecules have been shown to effectively inhibit these enzymes and exert pharmacological properties (Abbenante and Fairlie, Medicinal Chemistry, 2005, 1:71-104). Thus, the target molecule can be a protease inhibitor, such as the broad spectrum metalloproteinase inhibitor GM6001 (also known as Ilomastat or Galardin), which is not normally found in the body.

In another aspect, the invention includes a sample collection device. Another aspect of the invention includes a method for collecting a consistent sample, comprising contacting the sample collection device with a target medium in vitro or in vivo. Optionally, the diagnostic device of the invention can employ the sample collection device of the invention.

### BRIEF DESCRIPTION OF THE DRAWINGS

FIG. 1 shows an assay of the subject invention.

FIG. 2 shows a test sample of gelatin thin film digested with (A) 5 mg/ml, (B) 1 mg/ml, and (C) 0.1 mg/ml Pronase.

FIG. 3 shows components of capillary flow indicator strip and depictions of assays. (A) Emission filter, (B) capillary flow plates, (C) Sample pad, (D) excitation filter, (E) detection region, (F) conjugate pad, (G) region of capillary flow. Upon running the assay, the detection line can be verified against a standard scale to assess protease activity, as depicted in the “Top View” diagrams.

FIGS. 4A and 4B show drawings of an embodiment of the device of the invention using two color nanoparticle-coupled antibodies to two different target proteins. FIG. 1A shows the solid support, including the conjugate zone with chromogenic monoclonal antibodies (chromogenic Ab1 and Ab2) specific for target molecules 1 and 2 (Target 1 and Target 2), respectively, and immobilized monoclonal antibody (immobilized Ab3) specific for chromogenic A2; capture zone, including immobilized polyclonal antibodies (immobilized Ab1 and Ab2) specific to Target 1 and Target 2, respectively; and the direction sample flow. FIG. 4B shows the solid support after the solvent front has migrated from the sample pad, through the conjugate and capture zones, and to the control zone.

FIG. 5 shows an embodiment of the device of the invention, showing spectral color change for indicative of the ratio of target molecule 1 to target molecule 2.

FIGS. 6A and 6B show drawings of multi-lane embodiments of the device of the invention. FIG. 6A shows an embodiment that detects one target molecule and generates a relative standard color curve through the use of different samples of standard containing known levels of the target molecule, providing a visual (or fluorescent) gradient that will allow the relative level of target molecule to be measured in the sample.

FIG. 6B shows an embodiment that detects two different target molecules using two different antibodies and two different chromophores.

FIG. 7 shows a side view of one embodiment of the sample collection device of the invention.

FIGS. 8A-8C show top views of the sample collection device of the invention, dry (FIG. 8A); saturated, with opaque to translucent shift (FIG. 8B); and saturated, with color shift (FIG. 8C).

FIG. 9 shows a side view of one embodiment of the diagnostic device of the invention receiving a sample collection device of the invention, positioned in the sample receiving zone, interposed between a wicking zone and conjugate zone.

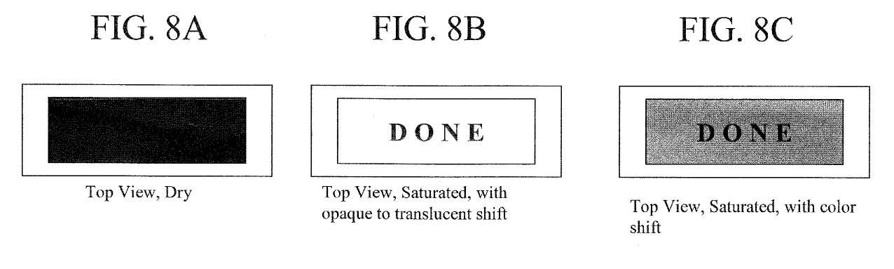

### BRIEF DESCRIPTION OF THE SEQUENCES

SEQ ID NO:1 is a peptide useful according to the subject invention.

SEQ ID NO:2 is a peptide useful according to the subject invention.

SEQ ID NO:3 is a peptide useful according to the subject invention.

SEQ ID NO:4 is a peptide useful according to the subject invention.

### DETAILED DESCRIPTION OF THE INVENTION

The present invention provides diagnostic methods and devices for detecting at least one analyte in a sample. The sample may be, for example, used an in vivo tissue sample or an in vitro sample (e.g., biological sample or environmental sample). The method and devices disclosed herein can be used to determine the presence, quantity, and/or concentration ratio of one or more target analytes. In one embodiment, the device provides an observable signal for use in real-time monitoring of the medium's molecular environment.

Advantageously, in certain embodiments, the subject invention provides assays that can be self-contained in a single unit. This facilitates conducting assays in the field and, in the case of healthcare, at the point of care.

The analytes detected according to the subject invention can be biochemical markers of health that can be used to direct therapy or prophylaxis. Thus, the assays of the subject invention can be used as part of a program to optimize treating and/or routing in a hospital.

The device and method of the invention can be of great benefit when diagnosing a pathological condition that has one or more biochemical markers. For example, a non-healing (chronic) wound is marked by the imbalance of several biological regulators, such as cytokines, proteases, and protease inhibitors, representing potential target analytes for the assays of the present invention. In one embodiment, the present invention is particularly useful for differential assays, in which a comparison between the amounts of multiple target molecules in the same sample or site is of interest.

In an embodiment that is specifically exemplified herein, the subject invention provides assays that can be used to determine and/or monitor the status of a wound. The assays are quick and easy-to-use. In specific embodiments, the assay can be carried out by, for example, a nurse utilizing either no instrumentation or only minimal instrumentation. In one embodiment, information about the status of a wound can be readily, easily and reliably generated in 30 minutes or less. In a preferred embodiment, the results are obtained in 15 minutes or less. Information about the wound can include, but is not limited to, protease activity, bacterial presence, and/or nitric oxide status.

With regard to protease activity, the activity of MMP-2, MMP-8, MMP-9 and elastase are of particular interest in wound care. In a specific embodiment, the assays of the subject invention are utilized to assess the status of chronic wounds. As used herein, reference to “chronic wounds” refers to wounds that after 2 weeks are not healing properly.

In a preferred embodiment, the subject invention utilizes a catalytic activity-based protease assay. This assay is advantageous because the pathogenic consequences of proteases are based on the activity of the proteases. This activity is difficult, if not impossible, to discern with molecular presence-based assays.

With regard to the assessment of bacterial presence at the site of a wound, the evaluation of the presence or absence of biofilm and/or specific bacteria such as MRSA are of primary importance. In the context of bacterial detection, an assay according to the subject invention can, for example, detect the presence or absence of penicillin binding protein in a method for determining whether MRSA are present.

A variety of assay formats can be used according to the subject invention. Particularly preferred assays are soluble substrate assays. These assays have been found to have favorable kinetic characteristics to facilitate easy, rapid and accurate detection of analytes. Particularly preferred assays as described herein include FRET and biotin anchor assays. Other assay formats, including those with a solid substrate may also be utilized as described herein.

A further assay format utilizes a thin film (similar to x-ray films) for the detection of enzymes such as collagenase. In this context of thin film can be, or can comprise, gelatin for the purpose of detecting collagenase. Alternative enzyme assays could utilize albumin or casein as the thin film.

The subject invention also provides sample collection methodologies which, when combined with the assays of the subject invention, provide a highly advantageous system for analyte evaluation in a wide variety of settings. In one embodiment, a “swab-in-a-straw” collection and assay system can be utilized as described herein.

The swab collection method is particularly advantageous for the evaluation of biofilm status as the swab is used to collect material that can include the matrix polysaccharides characteristic of biofilms.

### Applications for the Technology

The diagnostic devices and methods of the subject invention may be utilized in research and various industries, such as environmental management (e.g., water and wastewater treatment systems), bioremediation (e.g., to determine optimum conditions for microbial growth), public health (e.g., identification of rapidly growing infectious microbes), and homeland security (e.g., identification of rapidly growing bioterrorism agents).

Due to their ability to easily, quickly and accurately determine the presence, quantity, and/or concentration ratio of single or multiple target analytes, the devices and methods of the invention facilitate medical diagnoses at the physician's office and at the bedside of the patient. Ex vivo analysis of bodily fluids utilizing a device and method of the invention can be applied to a wide range of diagnostic tests. For example, potential applications include detection of licit and illicit drugs, detection of a wide range of biomarkers related to specific diseases, and detection of any other compounds that appear in bodily fluids. Analysis of bodily fluid samples using a device or method of the present invention can enable timely interventions for time-sensitive conditions or diseases.

The device and method of the invention can also be used in the area of chemical warfare, to assess the extent of exposure to sulfur mustard in the eyes, skin, and respiratory tract (e.g., lungs). The molecule(s) targeted for detection and/or measurement can be sulfur mustard reaction products such as alkylated serum proteins (e.g., albumin), alkylated hemoglobin, alkylated tear proteins (e.g., lactoferrin), alkylated epidermal proteins (keratins), alkylated lung fluid proteins, hydrolysis products of sulfur mustard in urine (thiodiglycol).

The device and method of the invention can be used for pulmonary applications, e.g., to assess the presence of respiratory infection. The molecule(s) targeted for detection and/or measurement can be those associated with viruses, fungi, or bacteria (e.g., viral, fungal, or bacterial antigens) that cause pulmonary infections, such as respiratory syncytial virus influenza virus, and pseudomonas.

The device and method of the invention can also be used for ocular applications, e.g., to assess the presence of ocular infection or molecules that are of diagnostic value in assessing infected and/or inflamed eyes. The molecule(s) targeted for detection and/or measurement can be protease inhibitors or molecules known to be associated with bacteria (e.g., pseudomonas or resistant bacteria) or viruses (e.g., adenovirus, Herpes simplex type I).

The device and method of the invention can be used for urological and/or gynecological applications, e.g., to assess the presence of urological and/or genital infections. The molecule(s) targeted for detection and/or measurement can be molecules known to be associated with pathogenic vaginal bacteria (e.g., beta hemolytic streptococci, pseudomonas), or viruses (e.g., herpes simplex type II).

The device and method of the invention can be used for obstetrical applications, e.g., to assess molecular risk factors for miscarriage or premature birth. The molecule(s) targeted for detection and/or measurement can be molecules known to be associated with premature rupture of membranes (PROM), such as matrix metalloproteinases (MMPs) and MMP inhibitors.

Another aspect of the invention concerns methods and devices for simultaneously detecting and measuring the relative amounts of multiple target molecules in a medium, or sample thereof, comprising contacting a device of the invention with the medium under conditions sufficient for the target molecules to be detected, if present. Preferably, the concentration of each target molecule is determined, relative to each other target molecule, and provided by a quantitative or semi-quantitative signal that is readily observable.

The application of the subject invention to wound care is described more fully below.

### Wound Care

The device and method of the invention can be used for dermal applications, e.g., to assess the presence of analytes in tissue or wound fluids that are of diagnostic value in assessing wound healing. The molecule(s) targeted for detection and/or measurement can be, for example, proteases, protease inhibitors, inflammatory cytokines, growth factors, molecules known to be associated with fungi and/or bacteria such as beta hemolytic streptococci, pseudomonas (e.g., bacterial antigens), resistant bacteria (e.g., MRSA, VRE, MRSE, and VRSA), or components of biofilms (and which are preferably unique thereto).

For example, the molecule(s) targeted for detection and/or measurement can be a penicillin-binding protein produced by MRSA (Berger-Bachi and Rohrer, Arch. Microbiol., 2002, 178:165-171).

The molecule(s) targeted for detection and/or measurement can be polysaccharides or glycoproteins that contribute to the formation of biofilms. Bacterial biofilms are highly heterogenous and found in the natural, industrial, and medical environments and include microorganisms embedded in a glycocalyx that is predominantly composed of microbially produced exopolysaccharide (Flemming et al., in “Biofilms: recent advances in their study and control”, 2000, pp. 19-34, Harwood Academic Publishers, Amsterdam, The Netherlands; Costerton et al., Science, 1999, 284:1318-1322; Costerton et al., J. Bacteriol., 1994, 176:2137-2142; Keevil et al., Microbiol. Eur., 1995, 3:10-14). The glycocalyx can provide protection against environmental change, such as antimicrobial agents, and may act as a reservoir for nutrients and ions (Allison, Microbiol. Eur., 1993, November/December: 16-19; Mah et al., Trends Microbiol., 2001, 9:34-39; Stewart and Costerton, Lancet, 2001, 358:135-138).

### Assays and Devices

The diagnostic devices of the present invention can be constructed in any form adapted for the intended use. Thus, in one embodiment, the device of the invention can be constructed as a disposable or reusable test strip or stick to be contacted with a medium for which knowledge of the molecular environment is desired (e.g., an anatomical site such as a wound site). In another embodiment, the device of the invention can be constructed using art recognized micro-scale manufacturing techniques to produce needle-like embodiments capable of being implanted or injected into an anatomical site for indwelling diagnostic applications. In other embodiments, devices intended for repeated laboratory use can be constructed in the form of an elongated probe.

The contacting step in the assay (method) of the invention can involve contacting, combining, or mixing the sample and the solid support, such as a reaction vessel, microvessel, tube, microtube, well, multi-well plate, or other solid support. Samples and/or binding agents of the invention may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., library members (candidate agent libraries). A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or -1536 well, plates (or trays). Optionally, binding agents may be immobilized on the solid support.

Optionally, the device of the invention includes an output device in communication with the sensing element of the device. An indication of a target molecule's presence or a detected target molecule's concentration can be displayed on the output device, such as an analog recorder, teletype machine, typewriter, facsimile recorder, cathode ray tube display, computer monitor, or other computation device. Optionally, in addition to the displayed presence of each target molecule or the concentration of each target molecule relative to each other, the output device displays the conditions under which the detection was carried out (such as temperature, salinity, time of day or night, etc.).

Optionally, in the various embodiments of the invention, the diagnostic method further comprises comparing the concentration of the target molecule in the medium (e.g., a bodily fluid), as determined above, to pre-existing data characterizing the medium (e.g., concentration of the same target molecule in the same patient or a different patient). The target molecule concentration may be that specific target molecule concentration observed under particular conditions.

Optionally, the method of the invention further comprises monitoring the presence and/or concentration of one or more target molecules in a medium over a period of time.

Simple “mix-and-read” assays minimize time and increase productivity; assays can be developed for naked eye or quantitative assessment using well established, relatively inexpensive detection technologies; easy-to-interpret detection system when used by non-technical personnel. In short, less equipment and fewer lab skills necessary to run the test.

### Substrate Cleavage Assay

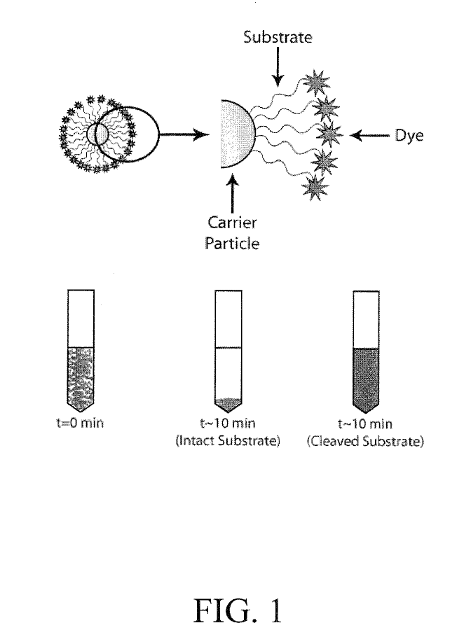

The enzymatic activity of proteases can be determined using substrate cleavage assays wherein a proteolytic activity of the sample is determined by monitoring the cleavage of a model peptide introduced into the sample. As depicted in FIG. 1, the system can comprise a microparticle having bound to its surface a large number of a dye-conjugated substrates. The microparticles are of sufficient density that, when dispersed in the assay solution, their settling rate is of the order of 5-10 minutes. The substrate is a natural or synthetic peptide sequence having a generic or highly enzyme-specific sequence. As such, the degree of enzyme specificity can be tuned to monitor the activity of a group of proteases or that of a single protease of interest. Finally, tethered to the substrate sequences are dye subunits which may be composed of single or multiple (e.g. dendritic, oligomeric, etc.) dye molecules conjugated to the free end of the substrate.

At t=0, the microparticles are exposed to the sample in a suitable assay buffer solution that is then mixed thoroughly to bring the particles into suspension. As the dense particles settle over the next 5-10 minutes, the proteases present in the sample cleave their substrate targets, thus allowing the dye molecules to enter solution and produce a detectable optical change of the assay solution.

If insufficient enzyme activity is present in the sample, the microparticles settle out of solution with their attached substrate-dye appendages and the assay buffer remains clear. The critical dye concentration required for the detection of sufficient enzymatic activity can be determined for a number of systems (i.e. naked eye or automated detection systems). Thus, the system is highly tunable for a number of single or multiplexed assays involving various critical enzyme concentrations of one or several proteases.

The proteolytic detection assays of the subject invention can be used to measure the protease levels in wound fluids, which is an indicator of anticipated healing or chronicity. Additionally, prior to attaching a graft or treating with a growth factor the nurse/doctor can ensure that the host environment is amenable to the graft/growth factor (i.e. that the graft/growth factor will not be destroyed).

### FRET Assay

The basis of the FRET assay is to bring a fluorescing dye close enough to a dye that prevents fluorescence (quencher) by coupling the dyes to a peptide that is a substrate for the protease being tested. Once the protease has severed the peptide the fluorescing dye can now separate far enough away from the quencher to produce a detectable signal.

The peptide joining the dye and quencher can be modified to produce specificity for the protease being measured. In a specific example, the DABCYL absorbs the color that EDANS fluoresces thereby preventing its detection.

In general, the mechanics for the quenching can vary depending on the dye and quencher combination, but the concept at the technological level remains the same. Once the peptide is cleaved the EDANS can separate far enough away from the DABCYL for the fluorescent color to escape and be detected.

Typically, a reaction between samples containing the protease of interest are mixed with these peptides and the reactions are continuously monitored by a fluorimeter for a change in fluorescent intensity. The products were quantified by measuring the fluorescence of a known quantity of the dye, and then scaled by the difference in fluorescence between free dye and the peptide fragment bound dye.

### PISA Assay

The PISA is similar to the FRET assay in that it employs a peptide that is selectively cleavable by the protease of interest, but it differs in how the cleavage event is conveyed to the user. In the FRET assay, while the peptide is linking the two dyes together, the fluorescence from the fluorescent dye cannot be detected. Once the peptide is cleaved, the two fragments can diffuse apart from one another allowing the fluorescent signal to be detected. Similarly, in the PISA, the peptide is linking a dye and an anchoring material (resin) which causes the dye to settle with the resin and therefore causes the solution to remain clear. Once the peptide is cleaved, the fragment with the dye can diffuse away from the anchoring resin causing the solution to change color.

In terms of what the protease interacts with (i.e. the peptide) nothing from the FRET is changed in the PISA. What has changed is how the signal is generated and read after the cleavage event and subsequent diffusion of the signaling dye molecule.

The FRET assay can be setup to be read as an all or nothing (good/bad) assay if a handheld excitation source (typically a blue pen light) is used. While in the PISA, the solution can be removed after the resin is settled, and it can be read by a spectrophotometer (either absorption or transmission) for quantification of the cleaved peptide (this is how both the FRET and thiopeptolide assays are read).

### Thin Film Assay

In one embodiment, the subject invention provides a rapid and simple method of assessing the protease activities in biological samples using a pigmented substrate thin film.

Various dyes, including Coomassie, readily bind to undigested proteins in solution. This phenomenon has been employed in routine laboratory techniques including sodium dodecyl sulfate polyacrylamide gel electrophoresis (SDS-PAGE) and zymography. In these laboratory methods, gels are stained to visualize electrophoretically separated proteins or regions of protein digestion by enzyme activity, respectively.

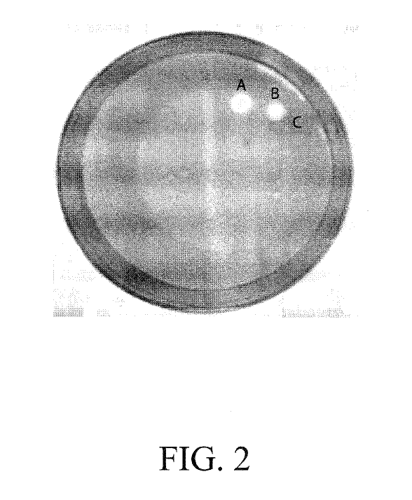

In accordance with the subject invention, chromo/fluorometrically labeled thin films of target substrates can be cast by a number of methods, including spin-coating, dip-coating, and tape-casting. Digestion of the target substrate can be visualized in minutes by simply reacting a volume of the biological sample onto the surface of the film and rinsing in water to remove the liberated dye and protease (FIG. 2). The film may be, for example, gelatin, albumin, casein, or fibrin.

### Fluorescence-Based Diagnostic Test Strip

The most sensitive of assays are those comprising fluorescently labeled markers for the detection of an activity of interest. Such assays are often capable of decreasing the detection threshold by orders of magnitude over their non-fluorescent counterparts. However, the ultra-sensitive detection of fluorescent species often requires specialized equipment that not only increases costs for the end user, but also limits the portability and versatility of the assay system. In one embodiment, the subject invention provides an ultrasensitive and simple fluorescence-based diagnostic test strip for the rapid detection of protease activity in various test specimens.

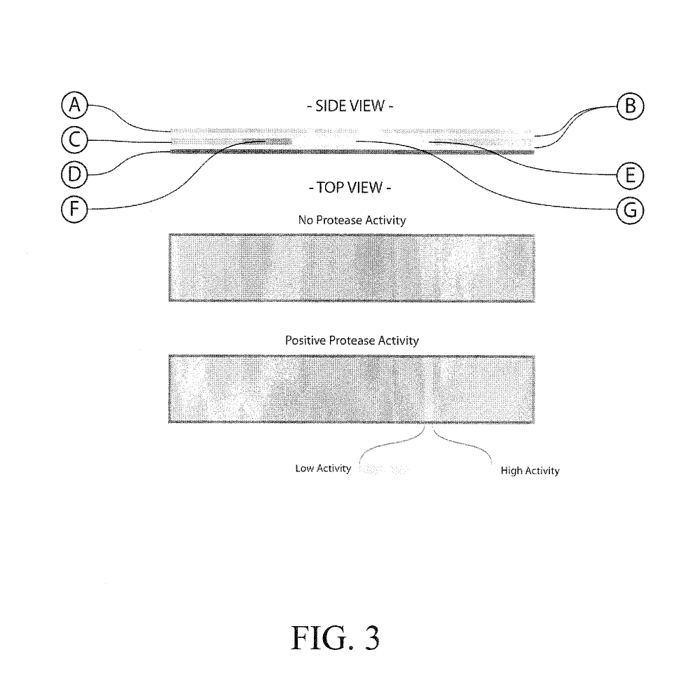

The components of the system are presented in FIG. 3. The key components are the pigmented, transparent excitation/emission filters and the biotin-labelled substrate.

The general structure of the substrate is Biotin-Fluorophore-Peptide′˜Peptide″ (Quencher). The substrate, as described here is based on fluorescence resonance energy transfer (FRET) chemistry. As such, any fluorescent signal emitted by the fluorochrome is absorbed by a “quencher” molecule in close proximity in the intact substrate. Upon cleavage of the substrate by the enzyme (protease) of interest, the fluorochrome and quencher are free to diffuse away from each other, thus allowing the fluorescence signal to be detected. Thus, when there is no cleavage of the substrate, no detectable signal is generated.

In one embodiment of the assay strip, the conjugate pad is loaded with lyophilized biotin-conjugated substrate. The test strip can be enclosed by transparent plates (polymer or glass) with sufficiently low-binding surface chemistry to ensure non-specific peptide/protein binding is negligible. These plates will thus sandwich the components of the test strip, leaving a capillary flow region in the central portion of the device. The detection region can be saturated with (strept)-avidin, thus binding the biotin-labelled end of the substrate as the fluid front flows from the sample/conjugate pads, through the capillary flow region, and towards the filter sink beyond the detection region. The detection region can comprise a 2-D line or 3-D porous matrix irreversibly conjugated with streptavidin.

The entire device can be encased in pigmented polymer films corresponding to the respective wavelengths of the excitation and emission maxima of the fluorescently labeled substrate. These filters allow the fluorescence of the digested substrate to be seen with the naked eye by simply holding the strip against a bright white light box such as those often employed for the visualization of x-ray photographs in the clinic.

### Lateral Flow Strip

In one embodiment the device of the invention can utilize lateral flow strip (LFS) technology, which has been applied to a number of other rapid strip assay systems, such as over-the-counter early pregnancy test strips based on antibodies to human chorionic gonadotropin (hCG). The device can utilize capture molecules (referred to herein as binding agents) target molecules. In one embodiment, one target molecule is a constant component of the medium (e.g., target tissue or sample), changing little in concentration (such as albumin in wound fluids), which is referred to herein as the “constant target molecule”; and another target molecule is one that changes concentration within the medium (such as a protease in wound fluids), which is referred to herein as the “variable target molecule”. Advantageously, the device and method of the invention can assess relative levels of multiple targets on a single solid support (e.g., strip).

The device can comprise a solid support with two or more binding agents, each binding agent having a molecular binding partner that represents a target molecule of interest. In one embodiment, the binding agents are monoclonal or polyclonal antibodies that are immuno-specific for the target molecules to be detected. In another embodiment, the binding agents are DNA aptamers that are specific for target nucleic acid molecules or other molecules to be detected.

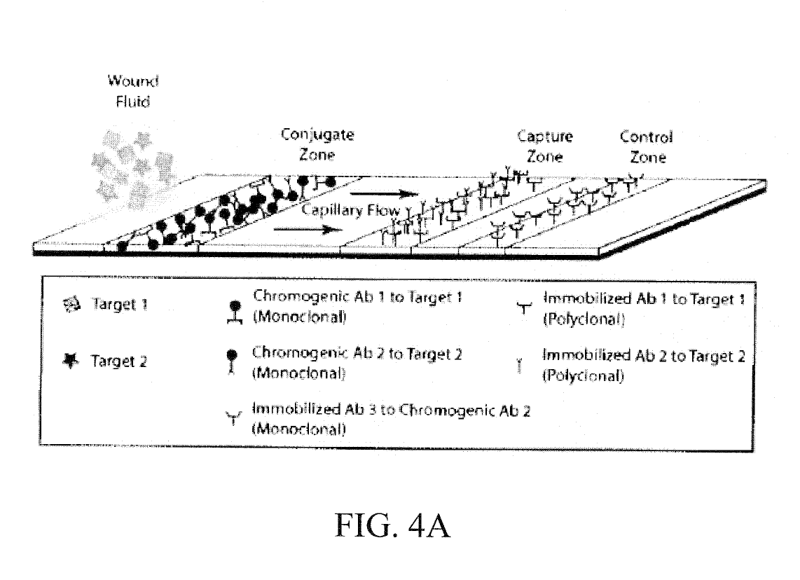

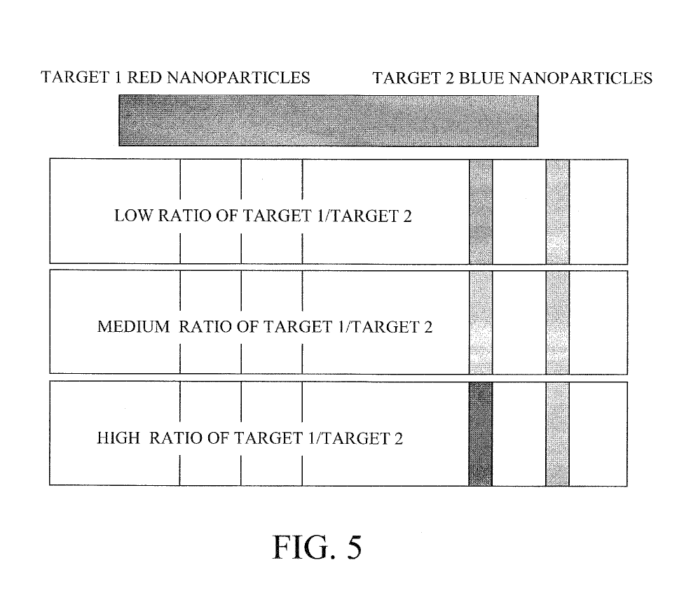

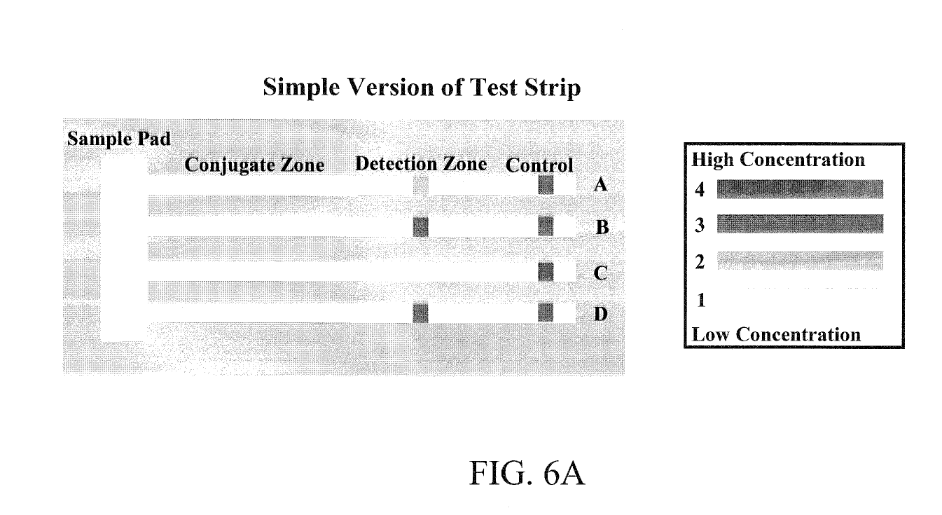

In certain embodiments, the device comprises a solid support (such as a strip or dipstick), with a surface that functions as a lateral flow matrix defining a flow path. The support comprises, in series, a number of zones (predefined areas): a medium (sample) receiving zone (on which a sample pad may be positioned); a conjugate zone; a capture zone (also referred to as a detection zone); and optionally, a control zone. Medium is contacted with the medium receiving zone (e.g., by placing a sample of the medium on the pad), and as the solvent front migrates (from left to right in FIGS. 4A and 4A), it carries the sample through the conjugate zone, which contains free (non-immobilized) binding agents (e.g., monoclonal antibodies or DNA aptamers) specific for different target molecules. Preferably, the binding agents are labeled with nanoparticles doped or otherwise associated with differently colored dyes (e.g., red and blue dyed nanoparticles). All of these components (potentially including binding agent-target molecule complexes and excess, and unbound binding agents) flow onto the capture zone, which contains immobilized binding agents (e.g., polyclonal antibodies) specific for the target molecules. Preferably, the binding agents immobilized in the capture zone are present in a 1:1 ratio. The nanoparticles will become fixed in the capture zone proportional to the concentration of the two or more target molecules, and the shade of color can be read to measure that ratio. Further migration of the solvent front (to the right in FIGS. 4A and 6A) will lead to the final developed result shown in FIG. 4B. The last zone (the control zone) contains immobilized binding agents (e.g., immobilized polyclonal antibody) specific for the binding agent (e.g., goat anti-mouse IgG) used to label one of the target molecules, and will serve as a positive control to show that active material (e.g., monoclonal antibody) was carried the full distance. An exemplified format for the device of the invention, including a control zone, capture zone, and conjugate zone, is shown in FIGS. 4A and 6A.

Preferably, the two or more binding agents are coupled to differently colored nanoparticles that will generate a spectrum of color (e.g., red to blue, with shades of purple), depending on the ratio of the variable target molecule and the constant target molecule in the medium. For example, if the binding agents are specific for matrix metalloproteinase-9 (MMP-9) and tissue inhibitor of matrix metalloproteinase-1 (TIMP-1), there are different colored nanospheres for MMP-9 and TIMP-1 (e.g., red for MMP-9 and blue for TIMP-1). Preferably, a ratio of nanospheres is immobilized at the capture zone, which will provide a signal representing the ratio of one target molecule to the other target molecule (e.g., MMP-9/TIMP-1), such as red or blue if enriched in one target molecule or the other target molecule. In the case of MMP-9 and TIMP-1, this will provide a read-out of a ratio shown to be significant in predicting wound healing (Ladwig et al., Wound Rep. Reg., 2002, 10:26-37).

In certain embodiments, the device of the invention comprises a solid support (such as a strip or dipstick), which functions as a lateral flow matrix defining a flow path. The support comprises, in series, a medium (sample) receiving zone on which a sample pad may be affixed; a conjugate zone; a capture zone (also referred to as a detection zone); and optionally, a control zone. A medium of interest is contacted with the medium receiving zone (e.g., by placing a sample of the medium on the pad), and as the solvent front migrates (to the right in FIGS. 4A and 6A), it carries the sample through the conjugate zone, which contains free binding agents (e.g., monoclonal antibodies or DNA aptamers) specific for different target molecules. Preferably, the binding agents are labeled with nanoparticles associated with differently colored dyes (e.g., red and blue dyed nanoparticles). All of these components (potentially including binding agent-target molecule complexes and excess, and unbound binding agents) flow onto the capture zone, which contains immobilized binding agents (e.g., polyclonal antibodies) specific for the target molecules. Preferably, the binding agents immobilized in the capture zone are present in a 1:1 ratio. The nanoparticles will become fixed in the capture zone proportional to the concentration of the two or more target molecules in the sample, and the shade of color can be read to measure that ratio. Further migration of the solvent front (from left to right in FIGS. 4A and 6A) will lead to the final developed result shown in FIG. 4B. The last zone (the control zone) contains immobilized binding agents (e.g., immobilized polyclonal antibody) specific for the binding agent (e.g., goat anti-mouse IgG) used to label one of the target molecules, and will serve as a positive control to show that active material (e.g., monoclonal antibody) was carried the full distance, through the zones of the support.

Preferably, the two or more binding agents are coupled to differently colored nanoparticles that will generate a spectrum of color (e.g., red to blue, with shades of purple), depending on the ratio of the variable target molecule and the constant target molecule in the tissue or sample. For example, if the binding agents are specific for MMP-9 and TIMP-1, there are different colored nanospheres for MMP-9 and TIMP-1 (e.g., red for MMP-9 and blue for TIMP-1). Preferably, a ratio of nanospheres is immobilized at the capture zone, which will provide a signal representing the ratio of one target molecule to the other target molecule (e.g., MMP-9/TIMP-1), such as red or blue if enriched in one target molecule or another target molecule.

Detection of target molecules and other assays carried out on samples can be carried out simultaneously or sequentially with the detection of other target molecules, and may be carried out in an automated fashion, in a high-throughput format.

The binding agents can be deposited but “free” (non-immobilized) in the conjugate zone, and are immobilized in the capture zone and control zone of the solid support. The binding agents may be immobilized by non-specific adsorption onto the support or by covalent bonding to the support, for example. Techniques for immobilizing binding agents on supports are known in the art and are described for example in U.S. Pat. Nos. 4,399,217; 4,381,291; 4,357,311; 4,343,312 and 4,260,678, which are incorporated herein by reference. Such techniques can be used to immobilize the binding agents in the invention. When the solid support is polytetrafluoroethylene, it is possible to couple hormone antibodies onto the support by activating the support using sodium and ammonia to aminate it and covalently bonding the antibody to the activated support by means of a carbodiimide reaction (yon Klitzing, Schultek, Strasburger, Fricke and Wood in “Radioimmunoassay and Related Procedures in Medicine 1982”, International Atomic Energy Agency, Vienna (1982), pages 57-62).

The binding agents of the conjugate zone are labeled. Preferably, these binding agents are labeled with chromogenic nanoparticles, which can be produced using known methods (Santra et al., Advanced Materials, 2005, 17:2165-2169, which is incorporated herein by reference in its entirety). Highly chromogenic nanoparticles can be generated by a reverse microemulsion method followed by sizing of the particles to select particles with desired diameters (e.g., in the range of 100 nanometers to 400 nanometers). The nanoparticles can be coupled to the binding agents using various chemical groups (—NH₂ being the preferred nucleophile). Because the capture zone contains immobilized target-specific binding agents in a predetermined ratio (e.g., a 1:1 mixture of two target-specific binding agents), the nan oparticles will become fixed in the capture zone proportional to the concentration of the two or more target molecules, and the shade of color can be read to measure that ratio.

The solid supports used may be those which are conventional for this purpose, constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. When, according to a preferred embodiment of the invention, the relative concentrations of target molecules in a tissue or body fluid are to be estimated without removing the tissue or body fluid from the body as a sample, the support should be one which is harmless to the patient and may be in any form convenient for insertion into an appropriate part of the body. For example, the support may be a probe made of polytetrafluoroethylene, polystyrene or other rigid non-harmful plastic material and having a size and shape to enable it to be introduced into a patient's mouth for estimation of steroids or other hormone concentrations in saliva, or into a patient's wound to determine the relative levels of proteases, protease inhibitors, or cytokines in the wound fluid. The selection of an appropriate inert support is within the competence of those skilled in the art, as are its dimensions for the intended purpose.

In one embodiment, the solid support has an absorbent pad or membrane for lateral flow of a liquid medium to be assayed, such as those available from Millipore Corp. (Bedford, Mass.), including but not limited to HI-FLOW PLUS membranes and membrane cards, and SUREWICK pad materials.

The amount of binding agent deposited on the solid support will be selected so as to meet the requirement for use of a trace amount relative to the fluid, as explained above. When the binding agent is to be introduced on the solid support into a patient's body the binding agent will naturally be one which is not harmful to the patient in the amounts used and under the conditions to which it is subjected in use (pH, etc.) and care will be taken to avoid the presence or retention of harmful substances in the body. The binding agent must as stated above be one which is specific to the analyte as compared to all other materials it is likely to encounter in use so that no interfering reaction or in-activation occurs but this obstacle is no different in principle from those faced in in vitro assays of body fluids and successfully solved. The choice of a binding agent satisfying these criteria is thus within the general competence of those skilled in the art. When the binding agent is deposited in an amount which is much less than the capacity of the support to adsorb or bond such agents it may be desirable to satisfy the remainder of the adsorption capacity of the support with a harmless protein or immunoglobulin or other inert material not reacting with the analyte nor harmful to the patient (if the solid support is to be inserted in the patient's body). Such materials and the means of applying them to the support are well known and standard methods can be used in this invention. The resulting support containing immobilized and/or non-immobilized binding agent can be stored in dry conditions under temperatures such as are known to be satisfactory for the storage of the known binding agents and will remain stable for extended periods of time, in the same way as commercially available hormone-measuring kits many of which already include hormone antibodies immobilized on a support.

### Nanoparticles

Nanoparticles of a variety of shapes, sizes and compositions have been successfully used in bioimaging, labeling and sensing (Medintz, I. L. et al. Nat. Mater., 2005, 4:435-446; Michalet, X. et al. Science, 2005, 307:538-544; Tan, W and Wang, K, Journal of Nanoscience and Nanotechnology, 2004, 4(6):559; Tan, W. et al. Med. Res. Rev., 2004, 24:621-638; Corstjens, P. L. A. M. et al. IEE Proc.-Nanobiotechnol., 2005, 152:64-72; Gao, H. et al. Colloid Polymer Sci., 2002, 280:653-660; Jain, T. K. et al. J. Am. Chem. Soc., 1998, 120:11092-11095; Zhao, X. et al. Adv. Mater., 2004, 16:173-176) due to their unique optical properties, high surface-to-volume ratio, and other size-dependent qualities, and may be utilized in making and using the diagnostic devices of the invention. With manipulated composition and surface modification, these nanoparticle probes have been able to enhance fluorescence signal, increase sensitivity, prolong detection time and generate better reproducibility.

Quantum dots (QDs) and dye-doped nanoparticles are representative fluorescent nanoparticle probes of increasing research interest. QDs are ultra-small (usually 1-10 nm in diameter), bright (20 times brighter than most organic fluorophores) and highly photostable, nanocrystalline semiconductors. Their broad excitation spectra, along with narrow, symmetric, size-tunable fluorescence emission spanning the ultraviolet to near-infrared, make them ideal for multiplex analysis (simultaneous detection of multiple analytes) without complex instrumentation and processing. Their high resistance to photobleaching and fair brightness make them appealing for long-term cellular and deep-tissue imaging (Medintz, I. L. et al. Nat. Mater., 2005, 4:435-446; Michalet, X. et al. Science, 2005, 307:538-544; Tan, W and Wang, K, Journal of Nanoscience and Nanotechnology, 2004, 4(6):559). However, QDs are difficult to make, the surface modification chemistry is still under investigation, the “blinking” characteristic (luminescence emission switches “on” and “off” by sudden stochastic jumps under continuous excitation) is a limiting factor for faster scanning systems such as flow cytometry, and cytotoxicity is a definite concern for in vivo applications (Medintz, I. L. et al. Nat. Mater., 2005, 4:435-446; Michalet, X. et al. Science, 2005, 307:538-544; Tan, W and Wang, K, Journal of Nanoscience and Nanotechnology, 2004, 4(6):559).

Another type of fluorescent nanoparticle probe that may be utilized is dye-doped nanoparticles, varying in size between 2-200 nm in diameter. With a large number of dye molecules housed inside a polymer or silica matrix, these nanoparticles give intense fluorescence signal that is up to 500 times that of QDs and 10,000 times that of organic fluorophores (Haugland, R. P. The Handbook: a Guide to Fluorescent Probes and Labeling Technologies, 10th edition, pp. 208-209). The extreme brightness makes them especially suitable for ultrasensitive bioanalysis without the need for additional reagents or signal amplification steps. Using dye-doped nanoparticle probes, a biomolecule recognition event is signaled by one or more nanoparticles, in which hundreds to thousands of dye molecules are integrated to greatly enhance the fluorescence signal. This signal enhancement facilitates ultrasensitive analyte/target determination and the monitoring of rare biological events that are otherwise undetectable with existing fluorescence labeling techniques. The polymer/silica matrix serves as a protective shell or dye isolator, limiting the effect of the outside environment (such as oxygen, certain solvents and soluble species in buffer solutions) on the fluorescent dye contained in the core of the particles.

Polymer or latex nanoparticles are commonly doped with fluorescent dyes following nanoparticle synthesis. A typical preparation method involves the swelling of polymeric nanoparticles in an organic solvent/fluorescent dye solution. The hydrophobic dye diffuses into the polymer matrix and is further entrapped when the solvent is removed from the particles through evaporation or transfer to an aqueous phase. The most common polymer matrices are polystyrene (PS), polymethylmethacrylate (PMMA), polylactic acid (PLA) and polylactic-co-polyglycolic acid (PLGA). Arrays of fluorescent polymer microspheres that differ in intensity, size or excited-state lifetime have also been extensively used in simultaneous assays to determine multiple analytes in a single sample (Stöber, W. et al J. Colloid Interface Sci., 1968, 26:62-69).

Silica nanoparticles doped with fluorescent dyes have also been used as labeling reagents for biological applications. Compared with polymer nanoparticles, silica nanoparticles possess several advantages: (i) Silica nanoparticles are easy to separate via centrifugation during particle preparation, surface modification and other solution treatment processes due to the higher density of silica (e.g., 1.96 g/cm³ for silica versus 1.05 g/cm³ for polystyrene); (ii) Silica nanoparticles are more hydrophilic and biocompatible, not subject to microbial attack and there is no swelling or porosity change with changes in pH (Zhao, X. et al. Adv. Mater., 2004, 16:173-176). (Polymer particles are hydrophobic, tend to agglomerate in aqueous medium and swell in organic solvents, resulting in dye leakage). Due to these advantages and the aforementioned fluorescence photostability over time and brightness, dye-doped silica nanoparticles have shown great promise in various biological applications (Corstjens, P. L. A. M. et al. IEE Proc.-Nanobiotechnol., 2005, 152:64-72), and may be utilized in the devices and methods of the invention.

There are two general synthetic routes for preparing dye-doped silica nanoparticles, the Stöber and microemulsion processes. In 1968, Stöber et al. introduced a method for synthesizing fairly monodisperse silica nanoparticles, with diameters ranging in size between 50 nm and 2 μm (Van Helden, A. et al. J Colloid Interface Sci., 1981, 81:354-368; Tan, C.; et al. J Colloid Interface Sci., 1987, 118:290-293; Coenen, S. and De Kruif, C. J. Colloid Interface Sci., 1988, 124:104-110; Van Blaaderen, A. and Kentgens, A. J. Non-Cryst. Solids, 1992, 149:161-178 (9). In a typical Stöber-based protocol, a silica alkoxide precursor (such as tetraethyl orthosilicate, TEOS) is hydrolyzed in an ethanol and ammonium hydroxide mixture. The hydrolysis of TEOS produces silicic acid, which then undergoes a condensation process to form amorphous silica particles. The details of the mechanism of Stöber-based nanoparticle formation have been extensively investigated (Van Blaaderen, A. et al. Langmuir, 1992, 8:1514-1517; Van Blaaderen, A. and Vrij, A. Langmuir, 1992, 8:2921-2931; Verhaegh, A. M. N. and Van Blaaderen, A. Langmuir, 1994, 10:1427-1438; Nyffenegger, R. et al. J. Colloid Interface Sci., 1993, 159:150-157) and the method has been optimized to synthesize dye-doped silica nanoparticles by covalently attaching organic fluorescent dye molecules to the silica matrix (Yamauchi, H. et al. Colloids Surfaces, 1989, 37:71-80; Osseo-Asare, K. and Arriagada, F. J. Colloids Surfaces, 1990, 50:321-339; Lindberg, R. et al. Colloids Surfaces A, 1995, 99:79-88. The procedure involves two steps: The dye is chemically bound to an amine-containing silane agent (such as 3-aminopropyltriethoxysilane, APTS), and then, APTS and TEOS are allowed to hydrolyze and co-condense in a mixture of water, ammonia, and ethanol, resulting in dye-doped silica nanoparticles. This approach enables the incorporation of a variety of organic dye molecules into the silica nanoparticles, which is advantageous for the present invention.

Dye-doped silica nanoparticles can also be synthesized by hydrolyzing TEOS in a reverse micelle or water-in-oil (W/O) microemulsion system, a homogeneous mixture of water, oil and surfactant molecules (Schmidt, J. et al. J. Nanoparticle Res., 1999, 1:267-276). In a typical W/O microemulsion system, water droplets are stabilized by surfactant molecules and remain dispersed in bulk oil. The nucleation and growth kinetics of the silica are highly regulated in the water droplets of the microemulsion system and the dye molecules are physically encapsulated in the silica network, resulting in the formation of highly monodisperse dye-doped silica nanoparticles (Santra, S. et al. Anal. Chem., 2001, 73:4988-4993; Santra, S. et al. J. Biomed. Opt., 2001, 6:160-166; Santra, S. et al. Langmuir, 2001, 17:2900-2906). In the last few years, a variety of dye-doped silica nanoparticles have been developed using the W/O microemulsion technique (Haugland, R. P. The Handbook: a Guide to Fluorescent Probes and Labeling Technologies, 10th edition, pp. 208-209; He, X. et al. J. Am. Chem. Soc., 2003, 125:7168-7169; Tapec, R. et al. J. Nanosci. Nanotechnol., 2002, 2:405-409; Qhobosheane, M. et al. Analyst, 2001, 126:1274-1278). To successfully entrap dye molecules inside of the silica matrix, polar dye molecules are used to increase the electrostatic attraction of the dye molecules to the negatively charged silica matrix, and the size of the dye molecules is larger than the pores of the silica matrix to prevent dye leakage. Water-soluble inorganic dyes, such as ruthenium complexes, can be readily encapsulated into nanoparticles using this method (He, X. et al. J. Am. Chem. Soc., 2003, 125:7168-7169; Wang, L. et al. Nano Lett., 2005, 5:37-43; Gerion, D. et al. J. Phys. Chem. B, 2001, 105:8861-8871). Leakage of dye molecules from the silica particles is negligible, probably due to the strong electrostatic attractions between the positively charged inorganic dye and the negatively charged silica. To synthesize organic dye-doped nanoparticles, various trapping methods have been employed, such as introducing a hydrophobic silica precursor (Qhobosheane, M. et al. Analyst, 2001, 126:1274-1278), using water-soluble dextran molecule-conjugated dyes and synthesizing in acidic conditions (Haugland, R. P. The Handbook: a Guide to Fluorescent Probes and Labeling Technologies, 10th edition, pp. 208-209A. These alternative methods aid in trapping hydrophobic dye molecules into the silica matrix. The unique advantage of the W/O microemulsion method lies in that it produces highly spherical and monodisperse nanoparticles of various sizes, and permits the trapping of a wide variety of inorganic and organic dyes as well as other materials such as luminescent quantum dots (Deng, G. et al. Mater. Sci. Eng. C, 2000, 11:165-172).

For biochemical assays and disease diagnosis, fluorescent dye-doped silica nanoparticles can be linked to the biorecognition elements (also referred to herein as binding agents), such as antibodies and DNA molecules. Many of these molecules can be physically adsorbed onto the silica nanoparticle surface. However, covalent attachment of biorecognition elements to the particle surface is preferred, not only to avoid desorption from the particle surface, but also to control the number and orientation of the immobilized biorecognition elements. To covalently attach the binding agent to the nanoparticles, the particle surface should be first modified with suitable functional groups (e.g., thiol, amine and carboxyl groups), as necessary. This is typically done by applying a stable additional silica coating (post-coating) that contains the functional group(s) of interest. For the Stöber nanoparticles, surface modification is usually done after nanoparticle synthesis to avoid potential secondary nucleation. Surface modification of microemulsion nanoparticles can be achieved in the same manner or via direct hydrolysis and co-condensation of TEOS and other organosilanes in the microemulsion solution (Santra, S. et al. Chem. Comm., 2004, 24:2810-2811; Santra, S. et al. Journal of Nanoscience and Nanotechnology, 2004, 4(6):590-599).

In addition to providing the reactive sites for conjugation with binding agents or other molecules, the functional groups also change the colloidal stability of the particles in solution. For instance, post-coating with amine-containing organosilane compounds neutralizes the surface negative charge of nanoparticles at neutral pH and hence reduces the overall charge of the nanoparticles. As a result, colloidal stability decreases and severe particle aggregation takes place in aqueous medium. To solve this problem, inert negatively charged organosilane compounds containing phosphonate groups or others are introduced as a critical dispersing agent during post-coating. Consequently, the nanoparticles possess a net negative charge and are well dispersed in aqueous solution (Zhang, M. et al. J. Am. Chem. Soc., 2003, 125:7790-7791; Farokhazd, O. C. et al. Cancer Res., 2004, 64:7668-7672). Other stabilization reagents, such as polyethylene glycol (PEG, a neutral polymer)-containing organosilane compounds, can also be added to the nanoparticle surface. The PEGylated surface is highly hydrophilic and enhances the aqueous dispersibility of the silica nanoparticles (Hermanson, G. T. Bionconjugate Techniques, Academic Press: San Diego, 1996). In addition, the PEGylated surface reduces non-specific binding by inhibiting the adsorption of undesired charged biomolecules.

After the nanoparticles are modified with different functional groups, they can act as a scaffold for the grafting of biological moieties (DNA oligonucleotides or aptamers, antibodies, peptides, etc.) by means of standard covalent bioconjugation schemes (Hilliard, L. R. et al. Anal. Chim. Acta., 2002, 470:51-56). For instance, carboxyl-modified nanoparticles have pendent carboxylic acids, making them suitable for covalent coupling of proteins and other amine-containing biomolecules using water-soluble carbodiimide reagents such as EDC (Deng, G. et al. Mater. Sci. Eng. C, 2000, 11:165-172). Disulfide-modified oligonucleotides can be immobilized onto thiol-functionalized nanoparticles by disulfide-coupling chemistry (Roy, I. et al. Proc. Natl. Acad. Sci. U.S.A., 2005, 102:279-284). Amine-modified nanoparticles can be coupled to a wide variety of haptens and drugs via succinimidyl esters and iso(thio)cyanates or proteins via NHS ester and carboxylic acid end groups. Other approaches use electrostatic interactions between nanoparticles and charged adapter molecules (Zhu, S. et al. Biotechnol. Appl. Biochem., 2004, 39:179-187; Ye, Z. Anal. Chem., 2004, 76:513-518) or between nanoparticles and proteins modified to incorporate charged domains. The bioconjugation or labeling strategy is rationally designed based on the biomolecular function of the surface-attached entities. For instance, protein recognition sites are oriented away from the nanoparticle surface to ensure that they do not lose their ability to bind to a target (Costa, A. R. C. et al. J. Phys. Chem. B, 2003, 107:4747-4755). After the bioconjugation step, the nanoparticles can be separated from unbound biomolecules by centrifugation, dialysis, filtration, or other techniques.

Sensitivity is a critical issue in modern biomedical research and disease diagnosis. The introduction of new fluorescent labels capable of high signal amplification is essential to addressing the growing need for highly sensitive bioassays. With numerous dye molecules trapped inside, dye-doped silica nanoparticles exhibit extraordinary signaling strength. For example, the effective fluorescence intensity ratio of one ruthenium bipyridine (RuBpy)-doped silica nanoparticle (Φ=60 nm) to one RuBpy dye molecule is 10⁴. Given the occurrence of self-quenching between dye molecules due to their close proximity inside the silica matrix, more than 10,000 dye molecules are presumed to be doped inside of a 60 nm nanoparticle. Thus, the impressive fluorescence properties of the nanoparticles can significantly lower the fluorescence detection limit in samples.

Photostability is a particularly important criterion for extended observation (from minutes to hours) of fluorescence signal under intense laser illumination. It is also especially useful for three-dimensional (3D) optical sectioning imaging, where a major obstacle is the photobleaching of fluorophores during acquisition of successive z-sections, which compromises the correct reconstruction of 3D structures. To demonstrate the high photobleaching threshold of nanoparticles, both nanoparticle and dye solutions were excited with a Xenon lamp and the emission intensities were monitored with respect to time. No noticeable photobleaching was observed for the dye-doped nanoparticles in solution for an hour, but the dye molecules lost 85% of the initial signal under identical conditions (He, X. et al. J. Am. Chem. Soc., 2003, 125:7168-7169). This observation proves that the silica coating isolates the dye molecules from the outside environment and thereby prevents oxygen penetration. In addition, when nanoparticles are employed for real biological sample imaging, the dye molecules are protected against degradation or photobleaching by the complex biological milieu because the silica matrix is highly resistant to chemical and metabolic degradation.

Moreover, whereas the organic fluorophores require customized chemistry for the conjugation of dye molecules to each biomolecule, the silica surface provides excellent versatility for different surface modification protocols. Since the nanoparticle surface can be functionalized with reactive end groups during synthesis, they can be readily modified with oligonucleotides, enzymes, antibodies, and other proteins. The nanoparticle-biomolecule complex can be used to express the activity of a desired process (e.g., immobilized enzymes) or can be used as affinity ligands to capture or modify target molecules or cells.

### Antibodies

Either member of the binding pair (the target molecule and binding agent) can be an antibody. Antibody molecules belong to the immunoglobulin family of plasma proteins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region. Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol., 1985, 186:651-666; Novotny and Haber, Proc. Natl. Acad. Sci. USA, 1985, 82:4592-4596).

The antibodies that are coupled (e.g., covalently) to the solid support can be monoclonal antibodies, polyclonal antibodies, phage-displayed mono-specific antibodies, etc. Preferably, the antibodies specifically bind to, or are immunospecific for, ligands that are part of, or attached to, an analyte of interest. Antibodies for detection of many analytes of interest are commercially available, or can be conveniently produced from available hybridomas, for example. Additionally, specific antibodies can be produced de novo using phage display or other protein engineering and expression technologies. Different antibodies that bind to different analytes can be utilized in a sensor of the invention.

An antibody that is contemplated for use in the present invention can be in any of a variety of forms, including a whole immunoglobulin, an antibody fragment such as Fv, Fab, and similar fragments, a single chain antibody that includes the variable domain complementarity determining regions (CDR), and the like forms, all of which fall under the broad term “antibody,” as used herein. The present invention contemplates the use of any specificity of an antibody, polyclonal or monoclonal, and is not limited to antibodies that recognize and immunoreact with a specific antigen.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments can retain an ability to selectively bind with the target molecule (e.g., antigen or analyte) and are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269 315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA, 1993, 90: 6444-6448.

The preparation of polyclonal antibodies is well known to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

The preparation of monoclonal antibodies likewise is conventional. See, for example, Kohler & Milstein, Nature, 1975, 256:495; Coligan et al., sections 2.5.1 2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1 2.7.12 and sections 2.9.1 2.9.3; Barnes, et al., Purification of Immunoglobulin G (IgG), in: Methods in Molecular Biology, Vol. 10, pages 79 104 (Humana Press, 1992).

The term “monoclonal antibody”, as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al., Proc. Natl. Acad. Sci., 1984, 81:6851-6855.

Methods of in vitro and in vivo manipulation of monoclonal antibodies are well known to those skilled in the art. For example, the monoclonal antibodies to be used in accordance with the present invention may be made by the hybridoma method first described by Kohler and Milstein, Nature, 1975, 256:495, or may be made by recombinant methods, e.g., as described in U.S. Pat. No. 4,816,567. The monoclonal antibodies for use with the present invention may also be isolated from phage antibody libraries using the techniques described in Clackson et al., Nature, 1991, 352:624-628, as well as in Marks et al., J. Mol Biol., 1991, 222:581-597. Another method involves humanizing a monoclonal antibody by recombinant means to generate antibodies containing human specific and recognizable sequences. See, for review, Holmes, et al., J. Immunol., 1997, 158:2192-2201 and Vaswani, et al., Annals Allergy, Asthma & Immunol., 1998, 81:105-115.

Methods of making antibody fragments are also known in the art (see for example, Harlow and Lane, Antibodies: A Laboratory Manual, Cold Spring Harbor Laboratory, New York, (1988), incorporated herein by reference). Antibody fragments can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab monovalent fragments. Alternatively, an enzymatic cleavage using pepsin produces two monovalent Fab fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H}, and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science, 1988, 242:423 426; Ladner et al., U.S. Pat. No. 4,946,778; and Pack, et al., Bio/Technology, 1993, 11:1271-1277.

Another form of an antibody fragment that may be used in the present invention is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick, et al, Methods: a Companion to Methods in Enzymology, 1991, Vol. 2, page 106.

Human and humanized forms of non-human (e.g., murine) antibodies may be used in the sensor and methods of the present invention. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies can comprise substantially all of at least one, and typically two, variable domains, in which all or substantially all of the CDR regions correspond to those of a non-human immuoglobulin and all or substantially all of the Fv regions are those of a human immunoglobulin consensus sequence. The humanized antibody optimally also will comprise at least a portion of an immunoglobulin constant region (Fc), typically that of a human immunoglobulin. For further details, see: Jones et al., Nature, 1986, 321: 522-525; Reichmann et al., Nature, 1988, 332:323-329; Presta, Curr. Op. Struct. Biol., 1992, 2:593-596; Holmes, et al., J. Immunol., 1997, 158:2192-2201, and Vaswani et al., Annals Allergy, Asthma & Immunol, 1998, 81:105-115.

### Aptamers

Aptamers have the capacity for forming specific binding pairs with virtually any chemical compound, whether monomeric or polymeric. Through a method known as Systematic Evolution of Ligands by EXponential enrichment, termed the SELEX process, it has become clear that nucleic acids have three-dimensional structural diversity not unlike proteins. One procedure for the selection of aptamers that bind to a desired target compound in accordance with the present invention is SELEX. SELEX is the in vitro evolution of nucleic acid molecules having highly specific binding ability to target molecules and is described in U.S. Pat. No. 5,475,096 (Gold and Tuerk); U.S. Pat. No. 5,270,163 (Gold and Tuerk); and WO 91/19813 (Gold and Tuerk), each of which is specifically incorporated by reference herein. These references describe methods for making an aptamer to any desired target molecule.

The SELEX process is based on the appreciation that nucleic acids have sufficient capacity for forming a variety of two- and three-dimensional structures and sufficient chemical versatility available within their monomers to act as ligands (form specific binding pairs) with virtually any chemical compound, whether large or small in size. The SELEX process involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the SELEX process includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

SELEX processes can be used to prepare aptamers for use with the device and method of the invention. The SELEX process enables the selection of nucleic acid molecules with specific structural characteristics, such as bent DNA. Other SELEX processes that can be used include, but are not limited to, the following: U.S. Pat. No. 5,580,737 (Polisky et al), which describes a method for identifying highly specific nucleic acid ligands able to discriminate between closely related molecules, which can be non-peptidic, termed Counter-SELEX; and U.S. Pat. No. 5,567,588 (Gold and Ringuist), which describes a SELEX-based method that achieves highly efficient partitioning between oligonucleotides having high and low affinity for a target molecule.

Aptamers with improved characteristics (such as improved in vivo stability or improved delivery characteristics) can be prepared using techniques that are known to those of ordinary skill in the art. For example, chemical substitutions at the ribose and/or phosphate and/or base positions can be performed to improve aptamer stability in vivo. Additional techniques for improving aptamer characteristics include those described in U.S. Pat. No. 5,660,985 (Pieken et al.), which describes oligonucleotides containing nucleotide derivatives chemically modified at the 5- and 2′-positions of pyrimidines.

Labeled dyes can be attached to an aptamer or other binding agent used in the device and method of the invention. The labeled dyes can be selected from many reactive fluorescent molecules that are known and readily available to those of skill in the art. Specific labeled dyes that are useful in practicing the invention include, but are not limited to, dansyl, fluorescein, 8-anilino-1-napthalene sulfonate, pyrene, ethenoadenosine, ethidium bromide prollavine monosemicarbazide, p-terphenyl, 2,5-diphenyl-1,3,4-oxadiazole, 2,5-diphenyloxazole, p-bis[2-(5-phenyloxazolyl)]benzene, 1,4-bis-2-(4-methyl-5-phenyloxazolyl)benzene, and lanthanide chelate. Preferably, pyrene is attached to the aptamer.

In certain embodiments, moieties such as enzymes, or other reagents, or pairs of reagents, that are sensitive to the conformational change of an aptamer binding to a target molecule, are incorporated into the engineered aptamers. Such moieties can be incorporated into the aptamer either prior to transcription or post-transcriptionally, and can potentially be introduced either into known aptamers or into a pool of oligonucleotides from which the desired aptamers are to be selected. Upon binding of the aptamer to a target molecule, such moieties are activated and generate concomitant signals (for example, in the case of a fluorescent dye an alteration in fluorescence intensity, anisotropy, wavelength, or FRET).

In one embodiment, the method of the invention is a method for simultaneously detecting the presence (or absence) of two or more different target molecules in a sample using a plurality of different species of aptamers as the binding agents, wherein each species of aptamer has a different moiety or label dye group, a binding region that binds to a specific non-nucleic acid target molecule, and wherein the binding regions of different aptamers bind to different target molecules; and a detection system that detects the presence of target molecules bound to the aptamers, the detection system being able to detect the different moiety or label dye groups.

The method can also be carried out with a plurality of identical aptamers. For example, each aptamer can include a moiety that changes fluorescence properties upon target binding. Each species of aptamer can be labeled with a different fluorescent dye to allow simultaneous detection of multiple target molecules, e.g., one species might be labeled with fluoroscein and another with rhodamine. The fluorescence excitation wavelength (or spectrum) can be varied and/or the emission spectrum can be observed to simultaneously detect the presence of multiple targets.

Binding agents other than antibodies or aptamers may be utilized, so long as there exists a molecular binding partner or specific binding partner (i.e., binding agent and corresponding target molecule), such that the binding agent undergoes detectable change(s) in physical properties in the presence of its binding partner (the target molecule). Molecular binding partners include, for example, receptor and ligand, antibody and antigen, biotin and avidin, and biotin and streptavidin. Thus, the binding agent and target molecule can together form a binding pair selected from the group consisting of antibody-antigen, enzyme-inhibitor, complementary strands of nucleic acids or oligonucleotides, receptor-hormone, receptor-effector, enzyme-substrate, enzyme-cofactor, glycoprotein-carbohydrate, binding protein-substrate, antibody-hapten, protein-ligand, protein-nucleic acid, protein-small molecule, protein-ion, cell-antibody to cell, small molecule-antibody to small molecule, chelators to metal ions, and air-born pathogens to associated air-bon pathogen receptors.

### DEFINITIONS

The terms “analyte” and “target molecule” are used interchangeably herein to refer to any component (molecular species) of a sample that is desired to be detected, or its influence or interaction detected or measured. The target molecule can be any substance for which a corresponding binding agent (its molecule binding partner) can be identified, such as a polypeptide, non-peptide small molecule, or biological agent, and can encompass numerous chemical classes, including organic compounds or inorganic compounds. The target molecule can be a substance such as genetic material, protein, lipid, carbohydrate, small molecule, a combination of any of two or more of foregoing, or other compositions. In some embodiments, the target molecule(s) are associated with bacterial, fungal, or viral infections (e.g., antigens). Target molecules can be naturally occurring or synthetic, and may be a single substance or a mixture. Target molecules can be or include, for example, an antibody, peptidomimetic, amino acid, amino acid analog, polynucleotide, polynucleotide analog, nucleotide, nucleotide analog, or other small molecule. A target polynucleotide can encode a polypeptide, or the target polynucleotide may be a short interfering RNA (siRNA), antisense oligonucleotide, ribozyme, or other polynucleotide that targets an endogenous or exogenous gene for silencing of gene expression.

The binding agent and target molecule can together form a binding pair, such as those selected from the group consisting of antibody-antigen, enzyme-inhibitor, complementary strands of nucleic acids or oligonucleotides, receptor-hormone, receptor-effector, enzyme-substrate, enzyme-cofactor, glycoprotein-carbohydrate, binding protein-substrate, antibody-hapten, protein-ligand, protein-nucleic acid, protein-small molecule, protein-ion, cell-antibody to cell, small molecule-antibody to small molecule, chelators to metal ions, and air-born pathogens to associated air-born pathogen receptors (e.g., air-born bacterial, fungal, or viral antigens).

Likewise, in some embodiments, two or more target analytes can have a molecularly competitive relationship (e.g., competing for the same receptor) or can be binding pairs, such as those selected from the group consisting of antibody-antigen, enzyme-inhibitor, complementary strands of nucleic acids or oligonucleotides, receptor-hormone, receptor-effector, enzyme-substrate, enzyme-cofactor, glycoprotein-carbohydrate, binding protein-substrate, antibody-hapten, protein-ligand, protein-nucleic acid, protein-small molecule, protein-ion, cell-antibody to cell, small molecule-antibody to small molecule, chelators to metal ions, and air-born pathogens to associated air-born pathogen receptors.

The target molecule can be a “biomarker”, which refers to naturally occurring and/or synthetic compounds, which are a marker of a condition (e.g., drug abuse), disease state (e.g., infectious diseases), disorder (e.g., neurological disorder, inflammatory disorder, or metabolic disorder), or a normal or pathologic process that occurs in a patient (e.g., drug metabolism). Biomarkers that can be detected using the device and method of the invention include, but are not limited to, the following metabolites or compounds commonly found in bodily fluids: acetaldehyde (source: ethanol; diagnosis: intoxication), acetone (source: acetoacetate; diagnosis: diet or ketogenic/diabetes), ammonia (source: deamination of amino acids; diagnosis: uremia and liver disease), CO (carbon monoxide) (source: CH₂Cl₂, elevated % COHb; diagnosis: indoor air pollution); chloroform (source: halogenated compounds), dichlorobenzene (source: halogenated compounds), diethylamine (source: choline; diagnosis: intestinal bacterial overgrowth); H (hydrogen) (source: intestines; diagnosis: lactose intolerance), isoprene (source: fatty acid; diagnosis; metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source: metabolism; diagnosis: periodontal disease/ovulation), MeS (source: metabolism; diagnosis: cirrhosis), Me₂S (source: infection; diagnosis trench mouth), alpha II-spectrin breakdown products and/or isoprostanes (source: cerebral spinal fluid, blood; diagnosis: traumatic or other brain injuries); prostate specific antigen (source: prostate cells; diagnosis: prostate cancer); and GLXA (source: glycolipid in Chlamydia; diagnosis: Chlamydia).

Additional biomarkers that can be detected using the device and method of the invention include, but are not limited to, illicit, illegal, and/or controlled substances including drugs of abuse (e.g., amphetamines, analgesics, barbiturates, club drugs, cocaine, crack cocaine, depressants, designer drugs, Ecstasy, Gamma Hydroxy Butyrate—GHB, hallucinogens, heroin/morphine, inhalants, ketamine, lysergic acid diethylamide—LSD, marijuana, methamphetamines, opiates/narcotics, phencyclidine—PCP, prescription drugs, psychedelics, Rohypnol, steroids, and stimulants); allergens (e.g., pollen, mold, spores, dander, peanuts, eggs, and shellfish); toxins (e.g., mercury, lead, other heavy metals, and Clostridium Difficile toxin); carcinogens (e.g., acetaldehyde, beryllium compounds, chromium, dichlroodiphenyltrichloroethane (DDT), estrogens, N-methyl-N′-nitro-N-nitrosoguanidine (MNNG), and radon); and infectious agents (e.g., Bordettella bronchiseptica, citrobacter, Escherichi coli, hepatitis viruses, herpes, immunodeficiency viruses, influenza virus, listeria, micrococcus, mycobacterium, rabies virus, rhinovirus, rubella virus, Salmonella, and yellow fever virus).

A “medium” or a “sample” of a medium can be any composition of matter of interest, in any physical state (e.g., solid, liquid, semi-solid, vapor) and of any complexity. The medium can be any composition reasonably suspecting of containing a target molecule that can be analyzed by the device or method of the invention. Typically, the medium is an aqueous solution or biological fluid. Samples can include human, animal, or man-made samples. The sample can be a biological sample (e.g., a bodily fluid, other biological fluid, or plant or seed material) or environmental sample (e.g., water, soil, sludge). Preferably, the sample is a fluid, such as a bodily fluid. The sample may be contained within a test tube, culture vessel, fermentation tank, multi-well plate, or any other container or supporting substrate. The sample can be, for example, a cell culture, human or animal tissue. Fluid homogenates of cellular tissues such as hair, skin and nail scrapings, meat extracts, skins of fruits, and nuts are biological fluids that may contain target molecules for detection by the invention.

The “complexity” of a medium or sample of a medium refers to the number of different molecular species that are present in the medium or sample.

The terms “body fluid” and “bodily fluid”, as used herein, refer to a mixture of molecules obtained from a human or animal subject. Bodily fluids include, but are not limited to, exhaled breath, whole blood, blood plasma, urine, tears, semen, saliva, sputum, nasal secretions, pharyngeal exudates, bronchioalveolar lavage, tracheal aspirations, interstitial fluid, lymph fluid, meningeal fluid, amniotic fluid, glandular fluid, sputum, feces, perspiration, mucous, vaginal or urethral secretion, cerebrospinal fluid, transdermal exudate, and wound fluid. Bodily fluid also includes experimentally separated fractions of all of the preceding solutions or mixtures containing homogenized solid material, such as feces, tissues, and biopsy samples.

The term “ex vivo.” as used herein, refers to an environment outside of a subject. Accordingly, a sample of bodily fluid collected from a subject is an ex vivo sample of bodily fluid as contemplated by the subject invention. In-dwelling embodiments of the device of the invention obtain samples in vivo.

A “patient” or “subject”, as used herein, refer to an organism, including mammals, from which biological samples can be collected (in vitro) or contacted (in vivo) to determine the relative levels of multiple target molecules in accordance with the present invention. Mammalian species that benefit from the diagnostic device and method of the invention include, and are not limited to, humans, apes, chimpanzees, orangutans, monkeys; and domesticated animals (e.g., pets) such as dogs, cats, mice, rats, guinea pigs, and hamsters.

The terms “molecular binding partners” and “specific binding partners” refer to pairs of molecules, typically pairs of molecules that exhibit specific binding to one another. Molecular binding partners include, without limitation, antibody-antigen, enzyme-inhibitor, complementary strands of nucleic acids or oligonucleotides, receptor-hormone, receptor-effector, enzyme-substrate, enzyme-cofactor, glycoprotein-carbohydrate, binding protein-substrate, antibody-hapten, protein-ligand, protein-nucleic acid, protein-small molecule, protein-ion, cell-antibody to cell, small molecule-antibody to small molecule, chelators to metal ions, and air-born pathogens to associated air-born pathogen receptors.

“Monitoring” refers to recording changes in a continuously varying parameter.

A “solid support” has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, tubing, spheres, containers, capillaries, pads, slices, films, plates, slides, etc., depending upon the particular application. Preferably, the solid support is planar in shape. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., HI-FLOW Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

A “coding sequence” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences.

As used herein, the term “polypeptide” refers to any polymer comprising any number of amino acids, and is interchangeable with “protein”, “gene product”, and “peptide”.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide”, “nucleic acid molecule”, and “nucleotide molecule” are used interchangeably herein and refer to a polymer of nucleotides joined together by a phosphodiester linkage between 5′ and 3′ carbon atoms. Polynucleotides can encode a polypeptide (whether expressed or non-expressed), or may be short interfering RNA (siRNA), antisense nucleic acids (antisense oligonucleotides), aptamers, ribozymes (catalytic RNA), or triplex-forming oligonucleotides (i.e., antigene), for example.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog” or “nucleic acid analog”, also referred to herein as an altered nucleotide/nucleic acid or modified nucleotide/nucleic acid refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. For example, locked nucleic acids (LNA) are a class of nucleotide analogs possessing very high affinity and excellent specificity toward complementary DNA and RNA. LNA oligonucleotides have been applied as antisense molecules both in vitro and in vivo (Jepsen J. S. et al., Oligonucleotides, 2004, 14(2):130-146).

As used herein, the term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA).

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “a microorganism” includes more than one such microorganism. A reference to “a molecule” includes more than one such molecule, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Following are examples that illustrate materials, methods, and procedures for practicing the invention. The examples are illustrative and should not be construed as limiting.

### EXAMPLE 1

### Diagnostic Antibodies Coupled to Nanoparticles Containing High Concentration of Fluorescent Dyes for Assessment of Wound Fluids

DNA aptamer technology with recently developed high color yield nanoparticle technology to create a test strip that can be placed in a chronic wound, and within minutes of absorbing wound fluid, enable the operator (e.g., a clinician) to visually assess the relative levels of key molecules that are diagnostic for good or poor wound healing. As shown in FIG. 4A, the basic test strip design will utilize lateral flow strip (LFS) technology, which has been applied to a number of other rapid strip assay systems such as over-the-counter early pregnancy test strips based on antibodies to hCG. General guides are available for developing LFS and are based on using products from filter or membrane companies such as Millipore and Pall. The test strip will use monoclonal and polyclonal antibodies that are specific for the two target molecules. A second method will utilize DNA aptamer chemistry to take advantage of the merits of aptamers relative to antibodies. Another unique property of the strip design will be combining two antibodies or aptamers on the same strip, one antibody or aptamer to detect the target molecule and the second antibody or aptamer to detect a molecule that is a constant component of wound fluids such as albumin. The two antibodies or aptamers will be coupled to differently colored nanoparticles, which will generate a spectrum of color (red to blue with shades of purple) depending on the ratio of the target molecule and the constant molecule in the wound fluid.

As shown in FIG. 4A, a sample of wound fluid is placed on the sample pad (far left) and as the solvent front migrates to the right, it carries the wound fluid over a zone with high concentrations of free monoclonal antibodies (or DNA aptamers) to the target molecules (conjugate zone), labeled with two different nanoparticles (e.g., red and blue dots). All of these components (including monoclonal antibody-antigen complexes and excess, unbound monoclonal antibodies) flow to the right onto the “capture zone” which is an immobilized 1:1 mix of polyclonal antibodies to the two target molecules. The nanoparticles will be fixed in this zone proportional to the concentration of the two target molecules, and the shade of color can be read to measure that ratio. Further migration of the solvent front to the right will lead to the final developed strip shown in the lower part of the figure. The last capture zone, called the “control zone”, contains immobilized polyclonal antibody specific to the type of monoclonal antibody used to label one of the molecules of interest (e.g., goat anti-mouse IgG), and will serve as a positive control to show that active material (monoclonal antibody) was carried the full distance.

### EXAMPLE 2

### Lateral Flow Strip Having Antibodies Specific to Diagnostic Proteins in Wound Fluids

In brief, a monoclonal Ab to the target MMP-9 (“M”) will be placed (but not immobilized) on the conjugate pad, as indicated in FIG. 4A. This will have bound, high-sensitivity nanospheres with a red dye droplet attached. When a sample of wound fluid is placed on the sample pad location on the porous membrane, it will migrate under capillary forces, to the conjugate pad and pick-up Ab from the large excess that is present there. The solvent front will continue to migrate until it reaches the polyclonal Ab to M, which is immobilized as a marker stripe on the “capture line”. Epitopes not covered by the first monoclonal antibody will be detected and bound by the immobilized polyclonal Ab, and will leave a dye/nanosphere mark behind as the solvent front passes through. This mark will be proportional to the concentration of M in the wound fluid, as long as there are more immobilized sites than there are molecules of M present.

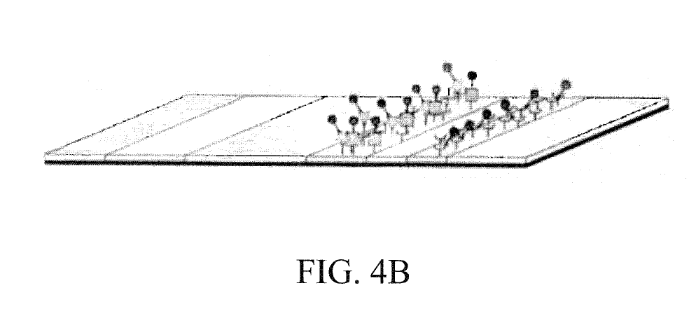

Because of the extreme selectivity of antibodies, it is possible to make a mixture of two monoclonal antibodies with different colored nanospheres for MMP-9 and TIMP-1; for example, red for MMP-9 and blue for TIMP-1. Both of these target antigens are large proteins (>50,000 D), and will have multiple epitopes per molecule, since a typical epitope is about 7-10 amino acids long. A ratio of nanospheres will be ultimately immobilized at the capture line, and will indicate the ratio of MMP-9/TIMP-1; red or blue in color, if enriched in one or the other (shown in FIG. 5). This will provide a read-out of the ratio needed to predict wound healing (Ladwig, G. P. et al. Wound Repair Regen, 2002, 10:26-37).

### EXAMPLE 3

### Consistent Sample Collection for a Lateral Flow Chromatographic Strip or Other Diagnostic Device

Neither absolute, nor relative protein level in a sampled fluid provides sufficient information to convey the chemical state, since it is the concentration that drives kinetics. To that end, the present inventors designed a sample collection device for consistently obtaining the same volume (within known tolerances) from sample to sample. With accurate volume information, the absolute and relative protein levels can be accurately interpreted (i.e., 1 μmol of protein in 100 μl volume is not the same situation as 1 μmol of protein in a 1 ml volume).

$${k}_{eq} = \frac{[ A ] \cdot [ B ]}{[ AB ]} = \frac{\frac{A}{Vol} \cdot \frac{B}{Vol}}{\frac{AB}{Vol}}$$

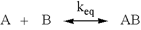

The sample collection device comprises an absorbent material (e.g., a pad) of any operative shape, backed with a saturation indicator and a semi-rigid, clear, material. Absorbent materials currently used in lateral flow chromatography have engineered bed volumes (total “empty” volume that can be occupied by the wicked fluid) with known tolerances that can provide estimable errors from sample to sample. These estimable input errors (deltas) can allow for estimable output errors (epsilons) in protein concentration determination (i.e., the protein's concentration is (X+/−epsilon).

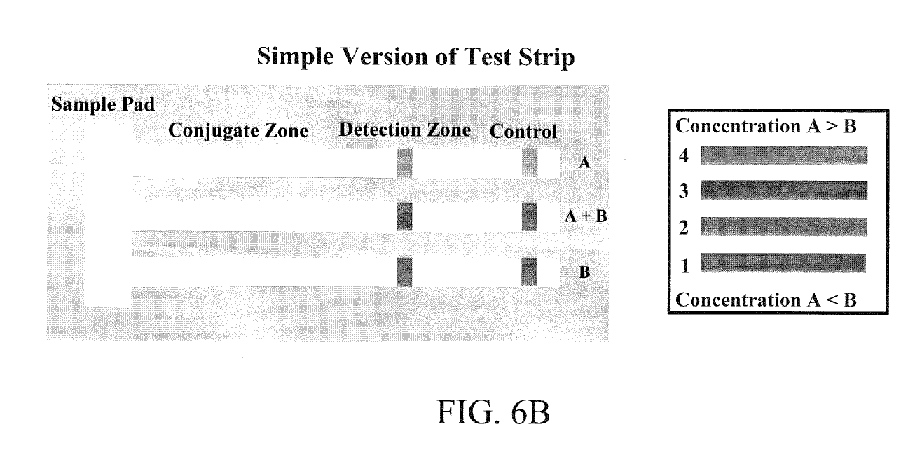

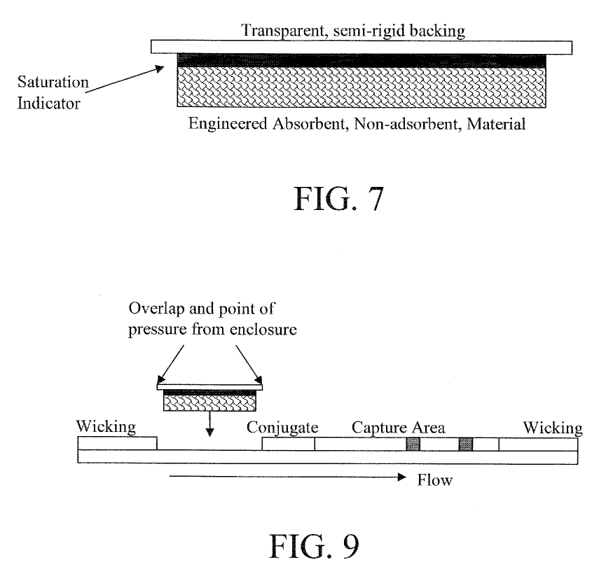

FIG. 7 shows a side view of one embodiment of the sample collection device of the invention. FIGS. 8A-8C show top views of the sample collection device of the invention, dry (FIG. 5A); saturated, with opaque to translucent shift (FIG. 8B); and saturated, with color shift (FIG. 8C).

The indicator is a substance that undergoes a chromogenic shift based on saturation, either from one color to another, or from opaque to translucent, for example. The transparent semi-rigid backing overhangs the sensor and absorbent, non-adsorbent, pad to allow for handling and to provide a point of contact for assembly into a housing device. The sample collection device can be driven by a buffer suitable to the application. Optionally, the diagnostic device of the invention can employ the sample collection device of the invention. FIG. 6 shows a side view of one embodiment of the diagnostic device of the invention receiving a sample collection device of the invention, positioned in the sample receiving zone, interposed between a wicking zone and conjugate zone.

### EXAMPLE 4

### Reference Standard for Protease Activity Measurement Device

In one aspect, the subject invention provides a transducer (or sensor). A sensor takes an input that changes the sensor and that change is considered an output. A sensor must be consistent, that is, it must have the same output for a given input. Also a sensor's output should be proportional to its input. Finally, because sensors are subject to unintended input, there is an expected difference between the output of equivalent inputs, or an error. The error should be predictable, and within a range that is acceptable to the system (highly dependent upon the application).

A novel device requires a standard to be compared against to demonstrate that it can accurately, and repeatedly ascertain the protease activity of a sample. There are several classes of assays that are currently in use in laboratories studying proteases and they can be categorized into two classes of tests, they either measure the presence of the protease, or they measure protease activity. In a preferred embodiment, assay of the subject invention is of the latter, since it will transform the enzymatic degradation of a peptide into a visible calorimetric signal.

An assay that is similar and quantitatively accurate is the cleavage of a FRET quenched fluorescent peptide (Matayoshi, E. D. et al. Science, 247 (February 1990): 954-958). The peptide is approximately 7 amino acids long and posses both a fluorescent dye and a quencher dye that, due to their proximity, “steals” the fluorophore's energy thus preventing a detectable signal upon illumination with an excitation light source. Once the peptide is cut, the two fragments can diffuse far enough apart for the fluorophore to be able to fluoresce. The strength of the photonic signal (i.e. the brightness of the light) is directly proportional to the amount of substrate cleaved and can be quantified by the use of standard photon counting equipment (fluorimeters, CCDs, etc. . . . ).

The width of the margin for acceptable error is wholly dependent upon how this device will be used. There are currently two ways the device will be employed, either as an indicator or as a diagnostic. In either case a standard test is needed as a reference.

The overall concept of an indicator is that it provides contextless information, a simple measurement devoid of judgment. For an assay to be an indicator it must indicate the level of protease activity without reference to an application (i.e. wound healing). To accomplish this, the device needs to be able to act as a sensor as described above and to indicate the protease activity present in any sample provided. The range of protease activities (e.g. 0 mg/ml-10 mg/ml equivalents) must be chosen as a design constraint. Upon completion of this test the individual using it would have a number that would be indicative of the amount of protease in the sample measured within some margin of error. Only a number is provided, the attending physician or other responsible individual would provide the judgment of what that number meant.

The assay must repeatedly measure protease activity with a consistent error. The FRET based assay can be used to determine whether the device is reporting the same MMP activity for any given sample. For example, taking an unknown amount of recombinant MMP-9 in a reaction buffer, splitting it in two, and exposing both assays to it. Additionally, in a separate reaction, the FRET assay can be run with a known quantity of recombinant MMP-9 as an internal standard (time control). After the assay has run for 10 min, the device will be read by eye and compared to the prepared visual standard and the FRET assay will be read on a plate reader. The internal control will be used to derive a fluorescence to MMP-9 ratio that can then be used to ascertain the amount of MMP-9 in the unknown FRET reaction. The results can then be compared and the errors calculated.

Alternatively, using the extinction coefficient for the fluorophore and the dye used in the device, the amount of unquenched fluorophore (FRET) or cleaved/soluble peptide (device) can be measured and compared using standard spectrophotometry.

### Diagnostic

A diagnostic on the other hand, pairs an indicator with a judgment; it is a program of sorts. By requiring that the device be binary (normal or problematic, low or high protease) the indicator (protease activity->color) is paired with a judgment (low or high). Reference to some clinical outcome sets the transition points (what protease level to go from clear “good” to saturated “bad”). For wound healing the thresholds can be, for example, Good, Intermediate, or Poor healers, as determined by wound closure rate, that correlate with (essentially) MMP-9 activity levels (MMP-9: TIMP-1 ratio, i.e. enzyme to inhibitor ratio) (Ladwig, G. P. et al. Wound Repair and Regeneration, 10 (2002): 26-37).

The standard assay can be used in the diagnostic to analyze the wound fluid to determine the triggering thresholds.

### EXAMPLE 5

### Kinetic Assessment of Two Immobilized MMP Substrates Labeled with QXL™ 610 Dye

### Introduction

QXL™ 610-conjugated substrate were analyzed for proteolytic cleavage and color generation visually and spectrophotometrically. The spectrophotometric data was used to construct a number of enzyme progress curves.

### Methods

Preparation of MMP Substrate Labeled with QXL™ 610 Dye

Substrate was prepared by solid phase synthesis using Fmoc amino acids and CLEAR-Base Resin (0.25 mmol, 0.65 mmol/g) using an automated peptide synthesizer (Applied Biosystems 43 1A). Synthetic conditions and coupling was performed according to the DCC/HOBt protocol provided by the manufacturer. Acetic anhydride was used to cap the peptide after each coupling step. An Fmoc-PEG2-Suc-OH spacer was coupled to the resin and the following peptide sequence was synthesized:

(SEQ ID NO:1)

QXL ™ 61 0-Lys-Pro-Gln-Gly-Leu-Glu-Ala-NH—CH₂—CH₂—
O—CH₂—CH₂—O—CH₂—CH₂—NH—COCH₂—CH₂—CO-Resin

70 umol resin and QXL™ 610 dye were combined with 10.7 mg HOBt, 15.17 mg HBtU, and 24.4 ul DIPEA and allowed to shake for 16 h. Following the reaction, the resin was filtered and washed in NMP, isopropanol, and dichloromethane. Deprotection was performed in 95% TFA/water for 60 min and the final product was washed in ethyl ether and dried.

### Assessment of MMP-9 Enzymatic Activity on Substrates

Stock MMP-9 (10 ug/ml) was prepared in MMP enzyme buffer (0.5% BSA, 0.1% Triton X-100 in ddH₂O). Substrate samples were dispersed in assay buffer (50 mM Tris-HCl, 50 uM ZnSO₄, 10 mM CaCl₂, 200 mM NaCl, 0.05% Brij₃₅, pH 7.5) using a large bore micropipette tip (10.4 mg/ml). Substrate, assay buffer, and MMP-9 were combined in microcentrifuge tubes and mixed gently by end-over-end mixing. MMP-9 (2 ug/ml, 200 ng/ml, 20 ng/ml) standards were reacted with the substrate (5 mg/ml) in a total reaction volume of 500 ul. Prior to each UV-Vis measurement, the mixtures were vortexed and centrifuged briefly and a 2 ul sample of the supernatant was analyzed on a NanoDrop ND-100 spectrophotometer.

### Screening of Protease Activities on Substrates

The substrates were screened for cleavage by trypsin, pronase, elastase, dispase, proteinase K in a similar manner as described for MMP-9. Briefly, 1 mg sustrate was combined with the enzymes in a 400 ul reaction. The reactions were incubated at 37° C. for 2.5 h until: color generation was noted.

### Kinetic Analysis of Pronase and Proteinase K Cleavage of Substrate AA

Substrate AA was reacted with pronase, proteinase K, and collagenase as previously described. Reactions were prepared with 10, 1, and 0.1 mg/ml enzyme and 1 mg substrate in 400 ul reaction tubes. All reactions were incubated at 22.5° C. for the duration of the experiment.

### Screening of Protease Activities on Substrates

Whereas none of the enzyme preparations were successful in cleaving the RH substrate, AA substrate reacted with pronase and proteinase K did produce a visible color within 2.5 h at 37° C. Pronase generated the most noticeable color change.

### Kinetic Analysis of Pronase and Proteinase K Cleavage of Substrate AA

Substrate incubation with pronase generated the most intense color throughout the course of the study. Visual detection of a color was first noted at approximately 90 min. By the second hour of incubation, a light blue hue was readily seen in the sample containing 10 mg/ml pronase. UV-Vis spectrograms were constructed to describe the increased color generation over the characteristic spectrum of the QXL™ 610. Generally, as the reaction progressed, an increase in absorbance was measured from 450-730 nm. A maximum absorbance intensity was observed at 598 nm.

Enzyme progress curves were constructed relating the cumulative absorbance (area under the curve and the absorbance at 598 nm vs. time. When the reaction was allowed to go to completion (10 mng/ml Pronase), a deep blue liquid was obtained. The substrate cleavage progressed in a nearly linear manner.

### EXAMPLE 6

### Investigation of the TNO211 Fluorescence-Based Assay as a Viable Rapid Detector of Protease Activity

In accordance with the subject invention, rapid and sensitive detection of metalloprotease activity is possible. In the process of conducting these studies, three key questions were addressed:
1) How fast can protease activity be detected using the TNO211 (Calbiochem #444256) solution-based fluorogenic peptide cleavage assay for MMP-2/-9 and how does this activity differ among MMP-2, MMP-9, and pronase?
2) Under what conditions is it possible to detect the cleavage of this substrate using a standard UV-source and the unaided eye?
3) What are the limits of detection, given an assay time of approximately 10-20 min, wherein an observer could easily distinguish between the presence or absence of MMP-2 and MMP-9 in a given sample?

### Methods

### Quantitation of MMP-2/-9 Using Substrate TNO211 (Calbiochem #444256)

The following is an assay that can be used to quantitate MMP-2/-9 activity in biological media.

Approach: A specific fluorogenic resonance energy transfer (FRET) peptide substrate with an MMP cleavable Gly-Leu bond and EDANS/Dabcyl as fluorophore/quencher combination. Useful for the detection of MMP activity [k_{cat}Kₘ=619,000 M⁻¹s⁻¹ for MMP-2, 206,000 M⁻¹s⁻¹ for MMP-9, 40,000 M⁻¹s⁻¹ for MMP-3, and 21,000 M⁻¹s⁻¹ for MMP-1; at 37° C., pH 7.64. Exhibits a high degree of sensitivity that is not affected by optical disturbances in biological media. Also useful for MMP activity measurements in synovial fluid and culture medium. Purity: ≧97% by HPLC. Excitation max.: ˜340 nm, Emission max.: ˜485 nm

### DABCYL-GABA-Pro-Gln-Gly-Leu-Glu(EDANS)-Ala-Lys-NH₂ (SEQ TD NO:2)

### Materials:

1. Substrate III (Calbiochem #444256, 500 ug): reconstituted to 1 mM in 377 uL1 DMSO.
2. Protease of choice
3. Enzyme Buffer (for the preparation of the protease standards): 0.1% Triton X-100, 0.5% BSA in PBS, pH 7-8

### 4. Substrate Buffers:

EDTA-free Buffer (for the determination of overall protease activity)
i) 50 mM Tris (pH 7.56), 200 mM NaCl, 5 mM CaCl₂, 50 uM ZnSO₄, 0.01M KH₂PO₄, 0.05% Brij₃₅

EDTA+Buffer (for the determination of non-MMP activity)
i) EDTA-free buffer+100 mM EDTA
   5. Opaque white 96-well fluorescence microtiter plate

### Methods:

1. Prepare a suitable standard curve diluted in enzyme buffer. Typically, a maximum of 50 ng/ml final protease concentration in the assay is used. Keep on ice until use.

2. Prepare the substrate solution by diluting the Stock Reconstituted Substrate (in DMSO) to 5.56 uM in the desired Assay Buffer (EDTA-free or EDTA-containing) to produce 90 ul of total EDTA-free or EDTA-containing Assay Buffer per well.

3. Pipette 90 ul substrate solution into each well to be assayed.

4. Take an initial fluorescence reading. To minimize interference due to the fluorescence of endogenous proteins in the samples, we routinely use excitation/emission wavelengths of 355/535 nm, respectively.

5. Pipette 10 ul standards, samples, or Enzyme Buffer (BLANK) into each well of the 96-well fluorescence assay plate.

6. Measure the change in fluorescence continuously until the standard range of interest is sufficiently resolved. Protect from light at RT or 37° C. between measurements.

7. Determine the best-fit curve relating the Change in Fluorescence (AF) vs.

[Protease]. Use this functional relationship to calculate the MMP activity equivalence in each of the samples.

### Study #1: Time-to-Detection of Protease Activity

One hundred, 10, 1, and 0.1 ng of pronase, MMP-2, MMP-9, and clostridial collagenase were assayed in a total reaction volume of 100 ul for cleavage of substrate TNO211 as detected by fluorescence. The reactions were monitored for approximately one hour after the addition of the proteases.

### SUMMARY

Standard curves were resolved as early as 3 minutes following reaction initiation. At higher protease concentrations (1 ug/ml), the pronase reaction reached completion within 13 minutes. Therefore, the succeeding comparisons were made for protease concentrations of 100 ng/ml and less. All reactions progressed in a concentration-dependent manner throughout the duration of the study. The substrate exhibited greater specificity for MMP-9 within the initial 30 minutes of the assay. For protease concentrations≦100 ng/ml, MMP-2, MMP-9, and collagenase activities were respectively 17±4%, 28±3%, and 2±1% of pronase's observed activity.

### Study #2: Determination of Reaction Conditions Necessary for Visual Detection of Protease Activity

The substrate was diluted serially in pH 7.5 and pH 9.0 assay buffer (500-0 uM). One microgram pronase was reacted with the various substrate solutions in a 96-well clear-bottom fluorescence microtiter plate. The plate was analyzed intermittently on a standard laboratory UV-box to observe the fluorescence intensity of the reactions. In addition, fluorescence measurements were taken as before and used to construct enzyme progress curves.

### SUMMARY

Significant fluorescence was observed within 6 minutes following the initiation of the reactions. Fluorescence intensities were greatest at approximately 100-200 uM TNO211. This was corroborated in the reaction progression curves, which clearly showed that the substrate was cleaved in a largely pH-independent manner. Furthermore, much of the reaction was complete within 20 minutes, as evidenced by a dramatic decrease in substrate velocity from this time point throughout the duration of the study.

### Study #3: Assessment of Protease Detection Sensitivity

To investigate the visual detection limits of pronase, standard curves (1000-0 ng) of the protease were reacted in 50, 100, and 200 uM substrate diluted in the assay buffer. Digital photographs were taken under white light and UV. These observations were compared to those obtained quantitatively using the fluorescence plate reader. The observed detection limits for pronase were used to calculate theoretical detection limits for MMP-2 and MMP-9 (17% and 28% as active as pronase, respectively).

### SUMMARY

The most relevant observations noted in this study are as follows: 125 ng pronase was detected within 10 minutes. This corresponded to approximately 735 ng and 450 ng of MMP-2 and -9, respectively. Of particular interest is the fact that these protease concentrations are on par with those of importance in our final detection kit. A 4-fold increase in sensitivity was observed by extending the assay time to approximately 20 minutes. All reactions generally reached equilibrium within 20 minutes.

### Study #4: Determination of the Optimum Concentrations of Na-Fluorescein and Rhodamine-B Necessary for a Solution-Based Fluorogenic Indicator Assay

Na-Fluorescein and Rhodamine-B were diluted serially from 20,000 ppm-2 ppb in assay buffer. The wells were photographed under UV light and the fluorescence intensity of Na-Fluorescein was measured using the fluorescence plate reader.

### SUMMARY

Substantial self-quenching was observed for both fluorogens at concentrations above 800 ppm. This phenomenon was less dramatic in the case of Na-Fluorescein which generally appeared brighter than Rhodamine-B. However, both fluorogens appeared to be most fluorescent at concentrations between 50 and 1000 ppm.

### EXAMPLE 7

### 5-FM Based TNO211 Peptide Assay

Part I of this example is a demonstration that pure recombinant MMP-9 generates a signal that is detectable within 10 minutes. Part II shows testing of MMP-9 spiked simulated wound fluid (fetal bovine serum, FBS) and a spiked uncharacterized wound vac fluid. While the vac fluid didn't produce a visually detectable signal even after 2 hours, the spiked FBS produced a signal that was unambiguously detectable by at least 23 minutes. Part III, includes the characterization of nearly 30 wound vac fluids that had been stored at −80° C. since about 2002. After finding samples that had a sufficient volume and protease levels characteristic of either high or normal/low protease activity levels, the wound fluids were exposed to the Anaspec peptide XV to determine whether genuine wound fluids with high versus low protease activity could be distinguished from one another in 10-20 minutes. The FRET peptide could produce a distinguishable signal by 15 minutes.

Part I: Testing with Pure Proteases

### Introduction

As a first step, the peptide is exposed to chronic levels of pure protease. This is done to limit the amount of potentially confounding variables, so that they can be identified as they arise (i.e. to eliminate ambiguity of negative results).

### Materials and Methods

MMP Assay

Buffer:

200 mM Tris, HCl pH 7.4

150 mM NaCl

CaCl

ZnCl

Brij 35

0.02% Azide

Peptide: Two vials of Anaspec, Inc.'s FRET peptide XV (1646.1 g/mol; 100 pg each; Sequence: QXL™520-y-Abu-Pro-Gln-Gly-Leu-Dab(5-FAM)-Ala-Lys-NH (SEQ ID NO:3)) were reconstituted with 60.7 pL of dimethyl sulfoxide (DMSO) to create a stock solution with a concentration of 1.0 mM. A 2× (50 pM) working solution was generated by diluting the 1.0 mM stock in MMP Assay Buffer 20-fold. Each reaction will be 20 pL at final volume requiring at least 0.5 pL of 1.0 mM stock, 9.5 pL of MMP assay buffer and 10 pL of sample per reaction.

Matrix Metalloprotease 9 (aka Gelatinase B): Recombinant active pure MMP-9 from Calbiochem (Cat# PF024; 83 kDa form) in a concentration of 100 ng/mL was used to create 40 pL working solutions at 2× concentration (4× reactions per concentration).

MMP-9 Dilutions for the 384-well plate

The concentrations used in the 384 well experiment reflect the final protease concentration in the reaction, not the in-wound protease concentration (multiply by 2).

The protease was ˜30 pL of 10 pg/mL MMP-9 (86 kDa form). The substrate was ˜60 μL of 50 μCM 5-FAM/QXL520 FRET Peptide (Fluorescein based TNO211). So the final protease concentration is ˜3.33 μg/mL with ˜33.3 μM.

### Plate Reader Settings:

The 384-well plate was read using a Wallac 1420 device and Wallac 1420 Explorer software. Briefly, the plate was orbitally shaken “fast” for 5.0 s, with a radius of 0.10 mm prior to being read. Two measurements were made per well (two different excitation wavelengths), first with the 355 nm excitation filter and second with 485 nm, both with an “Energy stabilized” “CW-lamp Energy” of 2600 and a measurement time of 0.1 s. The sample was read with the 535 nm measurement filter.

### Fluorescein Standard:

(M=332.306 g/mol) A serial dilution of pure fluorescein was generated with 4 replicates per concentration. The readings from the fluorimeter of this serial dilution will be used to estimate the number of cleavage events by equating the fluorescence levels in the standard to those fluorescent units gained as a consequence of de-quenching from cleavage of the peptide. Beginning with a stock solution of 2% w/v (20 g/L) the following concentrations were generated using the MMP Assay Buffer as the dilutent.

The data from the fluorimeter were imported into Microsoft Excel 2007 where they were averaged, graphed, and a trend line was determined. The equation from the trend line will serve as a map from fluorescence to number of cleavage products.

### Trialina 384-Well Plate:

Four replicates per protease concentration were plated out in a checkered pattern on the same plate as the fluorescein standard.

After 10 min, the plate was placed on an UV-transilluminator and imaged with a digital camera.

The plate was then placed in a fluorescent plate reader and read with both UV excitation and blue illumination 5 times with 10 minutes between the end of one complete read and the beginning of the next.

### Fluorescein Standard:

The replicates for each concentration were averaged for both the UV (355 nm) and blue (405 nm) excitation and two standard curves were generated for each excitation wavelength. The UV excitation generated a linear response whereas the blue excitation was parabolic. Only the samples in the standard below 25 μM fluorescein were used to generate a curve since this is the substrate concentration, and consequently the maximum fluorescing 5-FAM concentration.

### Trial in a 384-Well Plate:

The substrate was first tested with varying concentrations of pure MMP-9 protease. The fluorescein standard mentioned earlier was also run on this same plate.

### Trial in Centrifuge

After the plate-based validation, the leftover substrate and protease were used to test the system in a microcentrifuge tube. A signal visible with a handheld cyan led flashlight was present within 10 minutes. The signal was visible under normal lighting conditions, but the signal is enhanced with the lights out or with the tube shielded.

### Conclusions

Substrate “Anaspec XV” is capable of generating a signal within 10 minutes for pure MMP-9 activity near the threshold determined by Ladwig et al.

The signal is visible with a handheld cyan LED in normal lighting, but can be best seen when the tube is shielded from ambient light.

Standard curves can be based upon final substrate concentration to save time and increase gradations in the range where the test is likely to report. For instance, in this assay the maximum expected fluorescent signal would have been 25 μM of fluorescein.

The fluorescein appeared to generate a more consistent linear curve with UV excitation. The parabolic curve with the blue light excitation may be due to the excitation parameters set in the plate reader. Currently both UV and blue light had equal settings, since fluorescein is optimally excited by blue light, blue excitation energy can also be used.

Part II: Testing with MMP-9 Spiked Biofluids

### Introduction

The next step in testing the FRET peptide as a bedside diagnostic is to determine the interference caused by bulk proteins or other biomolecules. Two fluids, fetal bovine serum (FBS) and uncharacterized wound vac fluid (vac fluid), were spiked with enough recombinant MMP-9 to generate a final concentration of 10.0, 2.5 and 1.0 pg/mL or none at all (negative control).

### Materials and Methods MMP Assay Buffer:

200 mM Tris, HCl pH 7.4

150 mM NaCl

CaCl

ZnCl

Brij 35

0.02% Azide

### Peptides:

Anaspec, Inc.'s FRET peptide XV (1646.1 g/mol; 100 pg each; Sequence: QXL™520-y-Abu-Pro-Gln-Gly-Leu-Dab(5-FAM)-Ala-Lys-NH (SEQ ID NO:3)) were previously reconstituted with 60.7 pL of dimethyl sulfoxide (DMSO) to create a stock solution with a concentration of 1.0 mM. A 2× (50 pM) working solution was generated by diluting the 1.0 mM stock in MMP Assay Buffer 20-fold. Each reaction will be 20 pL at final volume requiring at least 0.5 pL of 1.0 mM stock, 9.5 pL of MMP assay buffer and 10 pL of sample per reaction.

In addition to Anaspec, Inc's FRET peptide XV, another FRET peptide with the same sequence (the “parent” peptide), but different fluorophore and quencher pair was used. A 1.0 mM stock solution (in DMSO) of the TNO211 peptide (Sequence: DABCYL-y-Abu-Pro-Gln-Gly-Leu-Glu(EDANS)-Ala-Lys-NH (SEQ ID NO:2)) was used in parallel for the sake of comparison.

### Matrix Metalloprotease 9 (aka Gelatinase B):

Recombinant active pure MMP-9 from Calbiochem (Cat# PF024; 83 kDa form) in a concentration of 100 ng/mL was used to spike FBS and an as of yet uncharacterized wound vac fluid. Recombinant MMP-9 was added until a final added concentration (above endogenous) of 10.0, 2.5, and 1.0 pg/mL.

Additionally, recombinant active pure MMP-9 from Calbiochem (Cat# xxxx; 67 kDa form) was used both as a positive control and to generate a MMP-9 activity standard for both FRET peptides. In order for the samples to have molar equivalent MMP-9 concentration the mass based concentration is scaled down by 80% (67 k/83˜=80%).

Plate Reader Settings: The 384-well plate was read using a Wallac 1420 device and Wallac 1420 Explorer software. Briefly, the plate was orbitally shaken “fast” for 5.0 s, with a radius of 0.10 mm prior to being read. For each peptide, two measurements were made per well. For the Anaspec FRET peptide XV, two excitation wavelengths were used, first using the 355 nm excitation filter and second with 485 nm. The sample was read with the 535 nm measurement filter. For the TNO211 peptide, the sample was excited with the same wavelength (355 nm), but read at two different wavelengths (460 nm and 535 nm). For all samples, the excitation was set to “Energy stabilized” “CW-lamp Energy” of 2600 and a measurement time of 0.1 s.

### Fetal Bovine Serum:

Low IgG

40 nm filtered

Cat. #: SH30151.03 Perbio HyClone

Lot #: ASG30077

Bottle #: 0153

Exp.: July 2012

### Fluorescein Standard:

The replicates for each concentration were averaged for both the UV (355 nm) and blue (405 nm) excitation and two standard curves were generated for each excitation wavelength. The UV excitation generated a linear response whereas the blue excitation was parabolic. Only the samples in the standard below 25 μM fluorescein were used to generate a curve since this is the substrate concentration, and consequently the maximum fluorescing 5-FAM concentration.

Two sets of two series were run on one plate, one spiked FBS, the other spiked vac fluid (very bloody), each tested with both the Anaspec XV and the original TNO211. The spiked vac fluid trial did not generate a visually detectable signal even after 2 hours with either peptide, whereas the spiked FBS generated as slightly detectable signal by 10 min with the 5-FAM-based peptide and an easily detectable signal by 30 min. The lack of signal from the vac fluid even in the presence of added recombinant MMP-9 suggests that there are high levels of endogenous inhibitor(s) (like TIMP-1).

### Trial in 600 μL Centrifuge Tubes:

The plate data demonstrated that using the spiked FBS as a pseudo wound fluid was feasible. Three 100 pL reactions (0, 1.0, and 10.0 pg/mL) of spiked FBS were run in pL centrifuge tubes. There was a noticeable difference amongst the tubes after 10 min.

### Conclusions

The spiked FBS demonstrated that an easily discernable signal can be read with standard lab illumination equipment in 10 min by eye or with cyan LED illumination by 28 min.

Part III: Testing with True Wound Vac Fluids

### Introduction

Thirty different wound fluid samples (presumably vac fluids) that were in −80° C. storage since ˜2002 were characterized using the two FRET peptides in a 382-well plate format in the same manner as Parts I & II. Samples found to have protease levels representative of chronic wounds and normal wounds were then used to test the ability of the FRET assay to generate a discernable signal in a short 10-30 min time frame.

### Materials and Methods Wound Vac Fluids:

Thirty vac fluids were found in −80° C. storage. The tubes were cataloged and photographed. Samples with less than 100 pL were either omitted or combined. The wound fluids were assigned arbitrary numbers (1-30). Vials with identical marks were treated as the same sample and the vials were also sub-numbered (i.e. 25(1), 25(2), etc. . . . ).

### Characterization of Wound Vac Fluids:

For each wound fluid with more than 100 pL, 3 replicates and 1 negative control (for background fluorescence) were plated. For those between 50-100 pL, a single measurement was made and single negative control was plated. Finally, a recombinant MMP-9 activity standard was generated by plating 4 replicates per concentration (0.0, 0.1, 0.5, 1.0, 2.5, and 5.0 pg/mL). 25 pM TNO211 was used as specified in Parts I & II. The standard curve generated with the recombinant MMP-9 was used to estimate the MMP-9 equivalent protease activity of the wound fluids. Finally, the estimated mass based concentration was scaled up by 1.25× since the standard was generated using the 67 kDa recombinant MMP-9 (i.e. 67 kDa->86 kDa).

Final Testing of the FRET Peptide with Wound Vac Fluids:

The Anaspec FRET peptide XV was used at a concentration of 50 pM in a 100 pL reaction (1:1 buffer and substrate to wound fluid) with a sample that has high protease activity and one that has low protease activity.

| Fr | Average MMP-9 Equiv. |
|---|---|
| #14 | 7.88 |
| #15 | 6.68 |
| #9 | 5.96 |
| #26 | 4.90 |
| #30 | 3.86 |
| #10 | 2.74 |
| #2 | 2.86 |
| #8 | 2.02 |
| #12 | 1.07 |
| #19 | 1.54 |
| #1 | 1.38 |
| #27 | 0.98 |
| #3 + 4 + 5 + 2 | 1.26 |
| #18 | 1.21 |

Samples #14 and #23 were chosen for the high and low wound fluid protease samples respectively.

### Conclusions

The 5-FAM based peptide can generate a signal that allows visual discernment between low protease and high protease levels within 15 min with a handheld cyan LED flashlight.

### EXAMPLE 8

### Testing Biotinylated Peptide #15

### Materials and Methods

The biotinylated fluoreceinated peptide #15 was reconstituted to a concentration of 10 mM in DMSO to serve as a stock solution. The entire mass was reconstituted because the lyophilized peptide formed a thin film that coated the walls of the glass vessel making it impossible to tare a small mass to be reconstituted. The 10 mM stock was further diluted to a 1 mM working stock. The final in-reaction concentration of substrate was 100 tM, which was chosen based on visual/fluorescent appearance of the diluted sample.

(SEQ ID NO:4)

Peptide #15:
Biotin-aAbu-Pro-Gln-Gly-Leu-Lys(5FAM)-Ala-Lys-NH₂

Pronase activity is similar to MMP-9 activity in that it can cleave TNO211 (albeit more rapidly). Three reactions were run, a negative control which contained no protease, a tube which contained 10 tg/mL, and a tube that contained 100 tg/mL.

All three tubes were shaken at room temperature (23° C.) for 30 min, then 30 μL of 500 mM EDTA was added to quench the metalloproteases. Then the reactions were immediately added to 10 kDa cut-off centrifuge filters to physically remove all proteases. This step required two separate filters per sample as the protein content was high enough to “clog” the filter after about half of the volume as filtered. The unfiltered volume was removed from the “clogged” filter and placed into a fresh filter.

Six handee spin columns were prepared in advance by loading 200 tL of the streptavidin agarose suspension (approximately 1:1 bead:buffer) and then centrifuging to remove the buffer. Upon completion of the protease removal step, the filtered volume was placed in a handee spin column loaded with ˜100 tL of a high-capacity streptavidin agarose and the volumes were well mixed by pipetting action. After the 3 samples had been passed through the first column, the samples were loaded on the three remaining fresh handee spin columns and pipette mixed once more. After centrifugation, there was enough of a difference between the negative control and the other samples to stop at this point, even through the negative wasn't completely filtered (an intrinsic problem with this type of assay to be discussed later).

The three samples were illuminated by UV transilluminator and photographed with and without filter the pictures provided are with the lights out, although the difference was still noticeable with the lights on.

Peptide (#15) was found to have favorable kinetics demonstrating that the hydrophobic rings of the dyes are causative of the rapid kinetics.

### CONCLUSION

The placement of fluorescein/dye at the P2′ position is causal of the rapid kinetics of the FRET peptides and previous constructs with Glu or Ala likely failed due to the lack of the big hydrophobic rings that both fluorescein and EDANS posses.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A device for simultaneously determining relative concentrations of multiple target molecules in a medium.

2. The device of claim 1, wherein said device utilizes binding agents specific for one or more target molecules and wherein the binding agents and target molecule(s) form a binding pair selected from the group consisting of antibody-antigen, enzyme-inhibitor, complementary strands of nucleic acids or oligonucleotides, receptor-hormone, receptor-effector, enzyme-cofactor, glycoprotein-carbohydrate, binding protein-substrate, antibody-hapten, protein-ligand, protein-nucleic acid, protein-small molecule, protein-ion, cell-antibody to cell, small molecule-antibody to small molecule, chelators to metal ions, and air-born pathogens to associated air-born pathogen receptors.

3. The device of claim 1, wherein the target molecules are each selected from the group consisting of a polynucleotide, polypeptide, lipid, carbohydrate, small molecule, antibody, peptidomimetic, amino acid, amino acid analog, polynucleotide analog, nucleotide, and nucleotide, or a combination of any of two or more of foregoing.

4. The device of claim 1, wherein the target molecules are each selected from the group consisting of a viral antigen, bacterial antigen, fungal antigen, sulfur mustard reaction product, protease, protease inhibitor, inflammatory cytokine, and growth factor.

5. The device of claim 1, wherein two of said target molecules are molecular competitors of one another.

6. The device according to claim 1, which utilizes enzymatic cleavage of a substrate in the detection of the enzyme.

7. The device of claim 1, wherein one of said target molecules is a known standard that is present in a medium to be screened for said target molecules.

8. The device of claim 1, wherein a dipstick is used as a solid support.

9. The device of claim 1, wherein a test strip is used as a solid support.

10. The device of claim 1, comprising a solid support that comprises a material selected from the group consisting of cellulose, polysaccharide, glass, polyacryloylmorpholide, silica, controlled pore glass (CPG), polystyrene, polystyrene/latex, polyethylene such as ultra high molecular weight polyethylene (UPE), polyamide, agarose, polyvinylidine fluoride (PVDF), polytetrafluoroethylene (PTFE; TEFLON), carboxyl modified teflon, nylon, nitrocellulose, and metals and alloys such as gold, platinum and palladium.

11. A method for detecting an analyte in a sample wherein said method is selected from the group consisting of:
a) a FRET assay;
b) an assay utilizing a thin film of substrate wherein digestion of the substrate by an analyte is visualized; and
c) a fluorescence-based diagnostic strip.

12. The method, according to claim 11, wherein the sample is an environmental sample.

13. The method, according to claim 11, wherein the sample is a bodily fluid.

14. The method, according to claim 11, wherein the sample is a bodily fluid, and wherein said method further comprises obtaining a sample of the bodily fluid from a subject.

15. The method, according to claim 11, wherein the sample is a bodily fluid selected from the group consisting of exhaled breath, whole blood, blood plasma, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, and wound fluid.

16. A method for evaluating the status of the healing process of a wound wherein said method comprises contacting a tissue or fluid sample obtained from the wound with a peptide that is cleaved by one or more proteases associated with wound healing, wherein if cleavage of the peptide occurs due to a protease in the sample, a detectable event occurs in less than 30 minutes from the time of contact.

17. The method, according to claim 16, wherein the detectable event occurs in less than 15 minutes.

18. The method, according to claim 16, wherein the detectable event can be observed without instrumentation.

19. The method, according to claim 16, which is used to detect at least one protease selected from the group consisting of MMP-2, MMP-8, MMP-9 and elastase.

20. The method, according to claim 19, wherein the peptide used to detect the protease is selected from the group consisting of SEQ ID NOS:1-4, and variants thereof.

21. The method, according to claim 16, wherein the assay format is a soluble substrate assay.

22. The method, according to claim 16, wherein the assay is a substrate cleavage assay.

23. The method, according to claim 16, wherein the detectable event is either the appearance or disappearance of fluorescence, or involves a qualitative and/or quantitative color change.

24. The method, according to claim 23, wherein the color change involves going from no color to some color.

25. The method, according to claim 16, which further comprises administering a treatment plan based on the results of the assay.

26. The method, according to claim 25, wherein said treatment comprises the administration of a protease inhibitor to the wound.

27. The method, according to claim 16, which is used to determine the relative concentration of multiple analytes.

28. The method, according to claim 16, wherein the assay comprises the use of biotin.

29. The method, according to claim 16, which further comprises detecting the presence of bacteria and/or fungi at the wound.

30. The method, according to claim 29, which comprises determining whether resistant bacteria strains are present.

31. The method, according to claim 16, wherein said method further comprises determining whether biofilm is present at the wound.

32. An assay device comprising a thin film of a substrate for an analyte.

33. The device, according to claim 22, wherein the analyte is an enzyme.

34. The device, according to claim 22, wherein the substrate is gelatin, albumin, casein, and fibrin.

35. The device, according to claim 22, wherein the thin film has been deposited by a method selected from the group consisting of spin-coating, dip-coating, or tape-casting.

36. As assay strip substantially as depicted in FIG. 3.
